(19) United States
(12) Patent Application Publication
(10) Pub. No.: US 2025/0161395 A1
(43) Pub. Date: May 22, 2025

(54) SPINAL CORD REGENERATION THERAPY

(71) Applicants: MOLEAC PTE LTD., Singapore (SG); UNIVERSITI KEBANGSAAN MALAYSIA, Selangor Darul Ehsan (MY)

(72) Inventors: Michel Maurice Jacques LAZDUNSKI, Nice (FR); Yogeswaran LOKANATHAN, Cheras, Kuala Lumpur (MY); Amaramalar Selvi NAICKER, Cheras, Kuala Lumpur (MY); Ohnmar HTWE, Cheras, Kuala Lumpur (MY); Ramesh KUMAR, Cheras, Kuala Lumpur (MY); Muhammad Da'in bin YAZID, Cheras, Kuala Lumpur (MY); Anam ANJUM, Cheras, Kuala Lumpur (MY)

(21) Appl. No.: 18/874,822

(22) PCT Filed: Jun. 26, 2023
(86) PCT No.: PCT/US2023/069050

§ 371 Date: Dec. 13, 2024

Related U.S. Application Data
(60) Provisional application No. 63/355,376, filed on Jun. 24, 2022.

(51) Int. Cl. A61K 36/69; A61K 36/232; A61K 36/236; A61K 36/286; A61K 36/481; A61K 36/537; A61K 36/65; A61K 36/736; A61K 36/882; A61P 25/00

(52) U.S. Cl. CPC A61K 36/69; A61K 36/232; A61K 36/236; A61K 36/286; A61K 36/481; A61K 36/537; A61K 36/65; A61K 36/736; A61K 36/882; A61P 25/00

(57) ABSTRACT

The present disclosure relates to spinal cord regeneration therapy. The disclosure, in one aspect, provides for the administration to a subject with a spinal cord injury of an effective amount of at least the four herbal components polygalae (thin leaf milkwort), astragali (membranous milkvetch), Ligusticum chuanxiong and Angelica sinensis (Chinese angelica), their roots or rhizomes, or extracts thereof. The disclosure, in other aspects, provides in vitro and in vivo methods for determining the effectiveness of drugs, and drug combinations, and the effective amount thereof required for treating a spinal cord injury.

### CROSS-REFERENCE TO RELATED APPLICATIONS

This application claims benefit of priority to U.S. Provisional Patent Application No. 63/355,376, filed Jun. 24, 2022, the disclosure of which is incorporated herein by reference in its entirety for all purposes.

### TECHNICAL FIELD

The present disclosure relates to a method for promoting spinal cord regeneration, and more particularly relates to inducing and/or accelerating recovery from a spinal cord injury by administering to a subject a herbal composition comprising a combination of traditional Chinese herbal medicinal products or extracts thereof.

### BACKGROUND

Spinal cord injury (SCI) is a serious medical problem. In severe cases, damage to the spinal cord may cause loss of connection between the brain and spinal cord, resulting in paraplegia, tetraplegia or even death. SCI often leads to devastating neurological deficits that not only disrupt the motor, sensory and autonomic functions, but also inflict loss of bladder and bowel control, respiratory problems, chronic pain and increased predisposition to infections. This results in a poor quality of life and a financial burden on SCI patients.

There is a lack of effective and safe therapeutic agents to treat individuals following SCI, despite it being a leading cause of death and disability. New therapeutic methods for treating SCI and associated neurological deficits are therefore urgently needed. More specifically, there is a need for new therapies that promote spinal cord regeneration and enable damaged or diseased nerves to function again.

### SUMMARY

The following summary is provided to facilitate an understanding of some of the innovative features unique to the present disclosure. A full appreciation of the various aspects of the present disclosure can be gained by taking into consideration the specification, claims, drawings, and abstract as a whole.

According to a first aspect, there is provided a method of treating a subject to induce and/or accelerate recovery from a spinal cord injury comprising administering to the subject an effective amount of a herbal composition comprising at least the four herbal components polygalae (thin leaf milkwort), astragali (membranous milkvetch), Ligusticum chuanxiong and Angelica sinensis (Chinese angelica), their roots or rhizomes, or extracts thereof.

In one embodiment of the first aspect, the herbal composition of the method further comprises at least one additional herbal component selected from the group consisting of salviae Miltiorrhizae (red sage), paeoniae rubra (red peony), flower of Carthamus tinctorius (safflower), Semen persicae (Prunus persica seed) and Acori tatarinowii (grassleaf sweetflag), their roots or rhizomes, or an extract thereof.

In one embodiment, the herbal composition of the method consists essentially of at least the four herbal components polygalae (thin leaf milkwort), astragali (membranous milkvetch), Ligusticum chuanxiong, Angelica sinensis (Chinese angelica), their roots or rhizomes, or extracts thereof.

In one embodiment, the herbal composition of the method consists essentially of the four herbal components polygalae (thin leaf milkwort), astragali (membranous milkvetch), Ligusticum chuanxiong, Angelica sinensis (Chinese angelica), their roots or rhizomes, or extracts thereof.

In one embodiment, the herbal composition of the method consists essentially of at least the nine herbal components polygalae (thin leaf milkwort), astragali (membranous milkvetch), Ligusticum chuanxiong, Angelica sinensis (Chinese angelica), salviae Miltiorrhizae (red sage), paeoniae rubra (red peony), flower of Carthamus tinctorius (safflower), Semen persicae (Prunus persica seed) and Acori tatarinowii (grassleaf sweetflag), their roots or rhizomes, or extracts thereof.

In one embodiment, the herbal composition of the method consists of a combination of extracts of the nine herbal components Radix polygalae, Radix astragali, Rhizome of Ligusticum chuanxiong, Radix Angelica sinensis, Radix et rhizoma salviae Miltiorrhizae, Radix paeoniae rubra, flower of Carthamus tinctorius, Semen persicae and Rhizoma Acori tatarinowii.

In one embodiment, the herbal composition of the method is a pharmaceutical composition. The pharmaceutical composition may also comprise one or more pharmaceutically acceptable carriers or excipients.

In a particular embodiment, the pharmaceutical composition for use in the method herein comprises a combination of extracts of the nine herbal components Radix polygalae, Radix astragali, Rhizome of Ligusticum chuanxiong, Radix Angelica sinensis, Radix et Rhizoma salviae Miltiorrhizae, Radix paeoniae rubra, flower of Carthamus tinctorius, Semen persicae and Rhizoma Acori tatarinowii together with one or more pharmaceutically acceptable carriers or excipients. In a specific embodiment, the pharmaceutical composition is MLLC901 (also known as NeuroAiD II™).

In another particular embodiment, the pharmaceutical composition for use in the method herein comprises a combination of extracts of the four herbal components Radix polygalae, Radix astragali, Rhizome of Ligusticum chuanxiong and Radix Angelica sinensis together with one or more pharmaceutically acceptable carriers or excipients. In a specific embodiment, the pharmaceutical composition is MLLC1501.

In one embodiment, the recovery from spinal cord injury is induced and/or accelerated by neuronal cell regeneration.

In one embodiment, the herbal composition of the method promotes the growth of neuronal cells.

In one embodiment, the neuronal cells are vertebrate neuronal cells.

In one embodiment, the vertebrate neuronal cells are human neuronal cells.

In one embodiment, the neuronal cells are derived from cortical neuronal cells.

In one embodiment, the administration of the herbal composition results in spinal cord regeneration and/or results in regeneration of a connection between the brain and spinal cord and/or results in regeneration of damaged neuronal tissue or cells.

In one embodiment, the administration of the herbal composition promotes limb motion recovery in paralyzed subjects.

In one embodiment, there is provided an effective amount of the herbal composition which is from about 1 mg/kg to about 100 mg/kg.

In one embodiment, there is provided a method that comprises administering a second agent used for treating a subject with spinal cord injury.

In one embodiment, the second agent is a pharmaceutical drug effective to control pain and/or muscle spasticity.

### BRIEF DESCRIPTION OF THE DRAWINGS

The accompanying drawings are included in order to more clearly illustrate specific embodiments of the present disclosure and the related art. The drawings included herein provide a further understanding of the disclosure, and are incorporated in and constitute a part of the present disclosure. It should be appreciated that the drawings illustrate implementations of the disclosure and, together with the rest of the disclosure, serve to explain the principles of the disclosure. It should be apparent that the drawings exemplify embodiments of the present disclosure, and a person having ordinary skill in the art may readily appreciate other embodiments from figures described herein.

FIG. 1 shows a flow diagram of the use of an in vitro spinal cord injury model in NSC-34 cell lines cultures where the injury is produced by mechanical (the “in vitro mechanical injury method”) and chemical (the “in vitro chemical injury method”) methods.

FIG. 2 shows a diagrammatic presentation of the in vitro mechanical injury method.

FIGS. 3a and 3b show flow diagrams of the use of in vivo spinal cord injury models in adult SD rats where the injury is produced by mechanical or chemical means.

FIG. 4 shows the results in the in vitro mechanical injury method following treatment with different concentrations of MLC901: a) Control cells with media only, b) 800 μg/mL, c) 1000 μg/mL, d) 1200 μg/mL and e) 1400 μg/mL

FIG. 5 shows a bar graph of the neurite outgrowth over 3 days following treatment with different MLC901 concentrations in the in vitro mechanical injury method.

FIG. 6 shows a bar graph of the mean neurite counts over 3 days following treatment with different MLC901 concentrations in the in vitro mechanical injury method.

FIG. 7 shows time lapse imaging over 3 days reflecting how MLC901 promotes neurite outgrowth and neurite count at the injured area in the in vitro mechanical injury method.

FIG. 8 shows time lapse imaging reflecting neurite outgrowth of injured NSC34 cells at Day 0 and Day 3 in the in vitro mechanical injury method.

FIG. 9 shows a bar graph of neurite growth after time lapse imaging at Day 0, Day 1, Day 2 and Day 3 with different MLC901 concentrations in the in vitro mechanical injury method.

FIG. 10 shows NSC-34 cell differentiation, reflecting motor neurons across 14 days of differentiation using 1 μM RA, 10× magnification.

FIG. 11 shows NSC-34 cell differentiation, reflecting motor neurons at Day 1, Day 3 and Day 5 of differentiation using 30 μM PGE2, 10× magnification.

FIG. 12 shows a bar graph reflecting % viability of differentiated NSC 34 cells to different MLC901 concentrations.

FIG. 13 shows an NSC-34 cell differentiation study at Day 7 using beta III tubulin and DAPI.

FIG. 14 shows ICC staining using DAPI and beta III tubulin in NSC-34 cells at Day 3 following treatment with different concentrations of MLC901.

FIG. 15 shows a bar graph reflecting percentage viability of NSC-34 cells to different MLC901 concentrations as compared to untreated cells.

FIG. 16 shows a protein signaling pathway and different protein markers involved in neuro-regeneration.

FIG. 17 shows expression of the protein markers p-AKT, P-GSKβ, ATF-3, GAP43, eIF2B and p-53 in untreated (UT) cells and cells treated with 1000 and 1200 μg/mL MLC901.

FIG. 18 shows a Western blot analysis of protein expression involved in PI3K/AKT neuro-regeneration pathways in untreated injured NSC34 cells, and cells treated with MLC901 at concentrations of 1000 and 1200 μg/mL.

FIG. 19 shows a bar graph reflecting expression of the PI3K/AKT pathway proteins phospho-Akt (Thr308), phospho-GSK-3β (Ser9) and ATF-3 using different MLC901 concentrations.

FIG. 20 shows a bar graph reflecting expressions of the PI3K/AKT pathway proteins GAP-43, p-53, and eIF2β using different MLC901 concentrations.

FIG. 21 shows a diagrammatic representation of the in vivo mechanical spinal cord injury model in adult SD rats using the calibrated forceps compression method.

FIG. 22 shows a locomotor BBB scale reflecting scores for joint movement, paw placement and toe clearance.

FIG. 23 shows line graphs of locomotor BBB scores in control (healthy) rats, untreated rats and rats treated with MLC901 3-28 days after mechanical spinal cord injury.

FIG. 24 shows a line graph of running wheel restraining ability of control (healthy) rats, untreated rats and rats treated with MLC901 after mechanical spinal cord injury.

FIG. 25 shows line graphs of a) time taken to walk a 0.5 m grid and b) distance travelled across a grid in 60 seconds for control (healthy) rats, untreated rats and rats treated with MLC901 after mechanical spinal cord injury.

FIG. 26 shows a line graph of an inverted grid test for control (healthy) rats, untreated rats and rats treated with MLC901 after mechanical spinal cord injury.

FIG. 27 depicts bar graphs reflecting the electrophysiology Somatosensory Evoked Potential (SEP) showing latency, duration, and amplitude in both MLC901 treated and untreated rats before and up to 4 weeks following mechanical spinal cord injury.

FIG. 28 shows the spinal cord morphology after extraction for control (healthy) rats, untreated rats and rats treated with MLC901 following mechanical spinal cord injury.

FIG. 29 shows a histopathological analysis of the extracted spinal cord with H and E staining for control (healthy) rats, untreated rats and rats treated with MLC901 following mechanical spinal cord injury.

FIG. 30 shows a transverse histopathological analysis of the extracted spinal cord with H and E staining for control (healthy) rats, untreated rats and rats treated with MLC901 following mechanical spinal cord injury.

FIG. 31 shows the timing of motor, electrophysiological and sensory assessments after KA injection in untreated (UT) and treated (T) animals relative to control (H) animals.

FIG. 32 shows in a) the BBB scores of control (H) and injured rats (UT and T) at pre-injury and after injury. The pre-injury and control (H) rats each received a BBB score of 21. The BBB score after injury in UT animals was significantly lower (p<0.05*) at days 7, 14, 21 and 28 compared to the BBB score after injury in T animals (n=5).

FIG. 32 shows in b) the distance covered by H, UT and T rats in 5 minutes. UT rats showed significantly reduced distances travelled at days 3, 7 and 14 (p<0.5)* and days 21 and 28 (p<0.1)** compared to T rats. H rats (p, 0.001***) travelled a significantly greater distance compared to both UT and T rats in each day of assessment (n=5).

FIG. 33 shows four locomotor assessments, namely a) grid walk, b) grid distance travel, c) running wheel and d) inverted grid. The T rats showed significantly better locomotor results (p<0.05*, p<0.01**) compared to UT rats (n=5, data presented as mean±SD).

FIG. 34 shows three somatosensory evoked (SEPs) assessments, namely a) amplitude, b) duration and c) latency in UT, T and H rats at pre-injury, day 14 and day 28 after injury. p<0.05*significance was observed in amplitude at days 14 and 28 between UT and T injured rats. The different waveforms d) for H, UT and T rats at day 14 post-injury are also shown. Data are expressed as mean±SD and was analyzed using one-way analysis of variance.

FIG. 35 shows H and E images of transverse sections of H, UT and T rats 28 days after injury. The image illustrates H and E stained sections. The darker stained area shows hemorrhage foci (pinkish color) containing pockets of erythrocytes (orange arrow). The appearance of lesion (blue arrows) is also observed in UT and T sections (progressive necrosis and cavitation). The different presentation of erythrocytes and neutrophils is indicated in the inflammation in UT and T samples. The image was taken at 4×, 10×, 20×, and 40× magnification with scale bar 500 um (n=5).

FIG. 36 shows immunohistochemistry images of a) antibody staining of transverse sections using GAP43, GFAP and DAPI and b) the fluorescence intensity of H, UT and T rats showing the expression of GAP43 (motor neuronal marker) as green fluorescence, GFAP (astrocyte and glial cells) as red fluorescence and DAPI (nuclear staining) as blue fluorescence. Intensive downregulation of GAP43 and GFAP in the UT group compared to T injury group results in more motor neural and astrocyte localization and damage. The images were taken by confocal microscope with 10× magnification and scale bar of 1000 um (n=5). Images were analyzed and compared by Image J software.

FIG. 37 shows the mechanism of action in KA-induced excitotoxicity in neuronal cells.

FIG. 38 shows anatomical location of the T13 thoracic spinal cord segment in top-down view of the back of the SD rat.

FIG. 39 shows the diagrammatic representation of the chemical injury model using kainic acid in which a) shows the marking of T11-T13 vertebrae, b) shows the subcutaneous incision, c) shows the exposure of muscle and spine, d) shows the removal of the muscle layer, e) shows the insertion of the syringe into the space between T12 and T13 vertebrae, f) shows the syringe placement at 45°, g) shows the slow perfusion of KA (0.1 uL/min) and h) shows the wound closure.

FIG. 40 a) shows the expression of AKT, p-AKT, p-GSK3β, GAP43, p53, ATF-3 and eIF2β detected via WB for C, UT and T rats after kainic acid excitotoxicity injury. b) Shows histograms of the relative quantitative expression of AKT, p-AKT, p-GSK3β, GAP43, p53, ATF-3 and elf2β. T rats showed higher expression of AKT, GAP43, p-53 (p<0.05*) and reduced expression of p-GSK3β (*p=0.05) relative to UT rats. There was no obvious difference in ATF-3 and elf2β expression between T and UT rats.

### GLOSSARY OF TERMS

This section is intended to provide guidance on the interpretation of the words and phrases set forth below (and where appropriate grammatical variants thereof).

The term “neuronal outgrowth” in the specification relates to the general directional outward growth of axons and dendrites. Neuronal outgrowth is important in synapse formation or development.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about” as used in relation to a numerical value means, for example, about ±30%, about ±20%, about ±10%, about ±5%, or about ±1% of the numerical value. Where necessary, the word “about” may be omitted from the definition of the invention.

The words “a”, “an” and “the” are employed to describe elements and components of the invention. This is done merely for convenience and to give a general sense of the invention. This description should be read to include one or at least one and the singular also includes the plural, unless the context clearly indicates otherwise. Thus, for example, the term “an agent” includes a reference to a single agent as well as a plurality of agents (including mixtures of agents). It should also be noted that the term “or” is generally employed in its sense including “and/or” unless the content clearly dictates otherwise.

The term “in vivo” as used herein includes a reference to using a whole, living organism. This contrasts with the term “in vitro” where a whole, living organism is not used. The term “in vitro” is to be understood as including, inter alia, “ex vivo” uses whereby cells, tissue etc. which does not form part of a whole, living organism may be employed (e.g. cells or tissues from cell or tissue cultures, biopsies, dead organisms etc.). Further non-limiting examples of “in vitro” relate to the use of cellular extracts or lysates.

The term “extraction” as used herein includes a reference to a method of separation in which plant material (e.g. chopped parts of a plant, whether fresh or dried) is contacted with a liquid solvent to transfer one or more components of the plant material into the solvent.

The terms “patient” and “subject” are used interchangeably herein and the terms include a reference to any human or non-human animal (preferably a mammal) that it is desired to treat using the present invention. However, it will be understood that “patient” or “subject” does not imply that symptoms are present. The term “mammal” as used herein refers to any member of the class Mammalia, including, without limitation, humans and non-human primates such as chimpanzees and other apes and monkey species; farm animals such as cattle, sheep, pigs, goats and horses; domestic/companion animals such as dogs and cats; laboratory animals (e.g. rabbits and rodents such as mice, rats, and guinea pigs, and the like). Preferably, the mammal is human.

The term “treatment” includes any and all uses which remedy a disease state or symptoms, prevent the establishment of disease, or otherwise prevent, hinder, retard, or reverse the progression of disease or other undesirable symptoms in any way whatsoever. Hence, “treatment” includes prophylactic and therapeutic treatment.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The terms “disease”, “disorder” and “condition” may be used herein interchangeably, unless the context clearly dictates otherwise.

RA means retinoic acid.

PGE2 means prostaglandin E2.

DAPI means 4′,6-diamidino-phenylindole.

BBB scale means the Basso, Beattie and Bresnahan locomotor rating scale, which is widely used to test behavioral consequences of spinal cord injury (SCI) in the rat.

SEP test means a somatosensory evoked potential test. The SEP test studies the relay of body sensations to the brain and how the brain receives those sensations.

H and E staining means hematoxylin and eosin staining, and is one of the principal tissue stains used in histology.

ICC staining means immunocytochemical staining, and refers to the staining of isolated or cultured intact cells where samples may be from tissue culture cell lines, either adherent or in suspension

ImageJ software is an open-source for processing and analyzing scientific images.

MTT assay means a mean transit time assay, and is used to measure cellular metabolic activity as in indicator of cell viability, proliferation and cytotoxicity.

DPBS means Dulbecco's phosphate-buffered saline.

DMEM/F12 means Dulbecco's modified eagle medium/nutrient mixture F-12.

FBS means fetal bovine serum.

AA means amino acid.

NEAA means non-essential amino acid.

KA means kainic acid.

Unless otherwise indicated, all technical and scientific terms used herein have the same meaning as commonly understood to one of ordinary skill in the art to which the invention belongs.

### DETAILED DESCRIPTION OF THE EMBODIMENTS

Detailed embodiments and implementations of the claimed subject matters are disclosed herein in detail with the technical matters, structural features, achieved objects, and effects with reference to the accompanying drawings as follows. It shall be understood that the disclosed embodiments and implementations are merely illustrative of the claimed subject matters which may be embodied in various forms. The present disclosure may, however, be embodied in many different forms and should not be construed as limited to the exemplary embodiments and implementations set forth herein. Rather, these exemplary embodiments and implementations are provided so that description of the present disclosure is thorough and complete and will fully convey the scope of the present disclosure to those skilled in the art. Specifically, the terminologies in the embodiments of the present disclosure are merely for describing the purpose of the certain embodiment, but not to limit the disclosure. In the description below, details of well-known features and techniques may be omitted to avoid unnecessarily obscuring the presented embodiments and implementations.

The configurations discussed in the following description are non-limiting examples that can be varied and are cited merely to illustrate at least one embodiment and are not intended to limit the scope thereof.

In one embodiment, there is provided a method of treating a subject to induce and/or accelerate recovery from a spinal cord injury comprising administering to the subject an effective amount of a herbal composition comprising at least the four herbal components Radix polygalae (root of thin leaf milkwort), Radix astragali (root of membranous milkvetch), Rhizome Ligusticum chuanxiong and Radix Angelica sinensis (root of Chinese angelica) or extracts thereof.

In one embodiment, the herbal composition of the method may further comprise at least one herbal component selected from the group consisting of Radix et Rhizome salviae Miltiorrhizae (red sage root), Radix paeoniae rubra (red peony root), flower of Carthamus tinctorius (safflower), Semen persicae (Prunus persica seed) and Rhizome Acori tatarinowii (rhizome of grassleaf sweetflag) or an extract thereof.

In one embodiment, the herbal composition of the method may consists essentially of at least the four herbal components Radix polygalae (root of thin leaf milkwort), Radix astragali (root of membranous milkvetch), Rhizome Ligusticum chuanxiong and Radix Angelica sinensis (root of Chinese angelica) or extracts thereof.

In one embodiment, the herbal composition of the method may consists essentially of at least the nine herbal components Radix polygalae (root of thin leaf milkwort), Radix astragali (root of membranous milkvetch), Rhizome Ligusticum chuanxiong, Radix Angelica sinensis (root of Chinese angelica), Radix et Rhizome salviae Miltiorrhizae (red sage root), Radix paeoniae rubra (red peony root), flower of Carthamus tinctorius (safflower), Semen persicae (Prunus persica seed) and Rhizome Acori tatarinowii (rhizome of grassleaf sweetflag) or extracts thereof.

In one embodiment, the herbal composition of the method consists of a combination of extracts of the nine herbal components Radix polygalae, Radix astragali, Rhizome of Ligusticum chuanxiong, Radix Angelica sinensis, Radix et Rhizoma salviae Miltiorrhizae, Radix paeoniae rubra, flower of Carthamus tinctorius, Semen persicae and Rhizoma Acori tatarinowii.

In one embodiment, the herbal composition of the method is a pharmaceutical composition. The pharmaceutical composition may also comprise one or more pharmaceutically acceptable carriers or excipients.

In a particular embodiment, the pharmaceutical composition for use in the method herein comprises a combination of extracts of the nine herbal components Radix polygalae, Radix astragali, Rhizome of Ligusticum chuanxiong, Radix Angelica sinensis, Radix et Rhizoma salviae Miltiorrhizae, Radix paeoniae rubra, flower of Carthamus tinctorius, Semen persicae and Rhizoma Acori tatarinowii together with one or more pharmaceutically acceptable carriers or excipients. In a specific embodiment, the pharmaceutical composition is MLLC901 (also known as NeuroAiD II™).

In another particular embodiment, the pharmaceutical composition for use in the method herein comprises a combination of extracts of the four herbal components Radix polygalae, Radix astragali, Rhizome of Ligusticum chuanxiong and Radix Angelica sinensis together with one or more pharmaceutically acceptable carriers or excipients. In a specific embodiment, the pharmaceutical composition is MLLC1501.

In one embodiment, the recovery from spinal cord injury is induced and/or accelerated by neuronal cell regeneration.

In one embodiment, the herbal composition of the method promotes the growth of neuronal cells.

In one embodiment, the neuronal cells are vertebrate neuronal cells. The vertebrate neuronal cells are human neuronal cells. The neuronal cells are derived from cortical neuronal cells.

In one embodiment, the administration of the herbal composition results in spinal cord regeneration and/or results in regeneration of a connection between brain and spinal cord and/or results in regeneration of damaged neuronal tissue or cells.

In one embodiment, the administration of the herbal composition promotes limb motion recovery in paralyzed subjects.

In one embodiment, there is an effective amount of the herbal composition which is from about 1 mg/kg to about 100 mg/kg.

In one embodiment, there is provided the method that comprises administering a second agent used for treating a subject with spinal cord injury.

In one embodiment, the second agent is a pharmaceutical drug effective to control pain and/or muscle spasticity.

NeuroAid II™, referred to herein as “MLC901”, is a composition containing extracts of nine herbal components (Radix astragali, Radix salvia miltiorrhizae, Radix paeoniae rubra, Rhizoma chuanxiong, Radix angelica sinensis, Carthamus tinctonius, Prunus persica, Radix polygalae and Rhizoma Acori tatarinowii). It is currently marketed as an oral treatment to support post-stroke recovery.

MLC1501 is a composition containing extracts of four herbal components (Radix astragali, Rhizoma chuanxiong, Radix polygalae and Radix angelica sinensis). It is currently in human clinical trials to assess efficacy in stroke recovery.

MLC901 and MLC1501, and the preparation thereof, are described in published PCT application number WO 2017/048191A1, the contents of which are incorporated by reference herein in their entirety.

In one embodiment, the herbal composition of the method herein consists essentially of the four herbal components Radix polygalae, Radix astragali, Rhizome Ligusticum chuanxiong and Radix Angelica sinensis or extracts thereof, together with any 1, 2, 3, 4 or 5 of the herbal components Radix salvia miltiorrhizae, Radix paeoniae rubra, Carthamus tinctorius, Prunus persica, and Rhizoma Acori tatarinowii or extracts thereof. Such herbal compositions, and the preparation thereof, are described in published PCT application numbers WO 2007/106049A1, WO 2010/053456A1, WO 2010/110755A1 and WO 2013/141818A1, the contents of which are incorporated by reference herein in their entirety.

A pharmaceutical composition herein may optionally comprise one or more pharmaceutically acceptable additives, carriers, and/or diluents. Examples of pharmaceutically acceptable additives include pharmaceutically acceptable excipients, buffers, adjuvants, stabilizers, diluents, fillers, preservatives, lubricants, or other pharmaceutically acceptable materials well known to those skilled in the art or as described herein. Examples of suitable pharmaceutical carriers or diluents include phosphate buffered saline solutions, water, emulsions (such as oil/water emulsions), various types of wetting agents, sterile solutions etc. Examples of excipients which may be employed include, for example, sugars, starches, celluloses, gums, proteins, dextrin and maltodextrin. Various formulations are commonly known and are thoroughly discussed in the latest edition of Remington's Pharmaceutical Sciences (Maack Publishing, Easton PA). In at least some embodiments, a pharmaceutical composition as herein described comprises an excipient, e.g. dextrin or maldextrin.

In one embodiment, a composition of the present disclosure (e.g. a pharmaceutical composition) may be comprised within a kit. The kit may, in addition to the herbal components, comprise instructions for use. The kit may be promoted, distributed, and/or sold as a unit for performing one of the aspects of the present disclosure.

In general, pharmaceutical compositions of the present disclosure may be prepared according to methods known to those of ordinary skill in the art.

The compositions may, for example, be a solution, a suspension, liquid, chopped herbs, powder, a paste, aqueous, non-aqueous or any combination thereof.

The compositions (e.g. pharmaceutical compositions) of the present disclosure may be administered by any suitable route, such as orally, parenterally, intravenously, subcutaneously, intradermally, intraperitoneally or topically, in liquid, semi-liquid or solid form and are formulated in a manner suitable for each route of administration. The term “administering” and variations of that term including “administer” and “administration”, includes contacting, applying, delivering or providing a composition of the present disclosure to an organism, or a surface by any appropriate means.

The herbal components may be administered in a therapeutically effective amount (either as a single dose or as part of a series of doses). By an “effective amount” or a “therapeutically effective amount” is meant the amount administered to achieve physiological significance. An agent is physiologically significant if it is present in an amount that results in a detectable change in the physiology of a recipient patient such that beneficial or desired results are achieved.

The exact amount required will vary from subject to subject depending on factors such as the species being treated, the age, weight and general health of the subject, the condition being treated and the severity of the condition, the mode of administration, the gender of the subject, diet, time and frequency of administration, drug combination(s), and tolerance/response to therapy and so forth.

In one embodiment, compositions (e.g. pharmaceutical compositions) of the present disclosure are administered as capsules taken one or more (e.g. 1, 2, 3 or 4) times per day. In a particular embodiment, MLC901 is administered in the form of 2 capsules, taken 3 times per day. In a particular embodiment, MLC1501 is administered in the form of 4 capsules, taken 2 times per day. For patients with swallowing difficulties, capsules may be opened and powder diluted in water that can be drunk as such or injected via a gastric tube.

The duration of treatment is typically 3 or more months, adaptable with regard to the patient's condition. In one embodiment, the patient's daily dose is about 500 mg to about 8 g, or about 1 g to about 8 g (e.g. about 1 g, 2 g, 3. g, 4 g, 5 g, 6 g, 7 g or 8 g). A “daily dose” can be a single unit dose or multiple unit doses (of tablets, capsules etc.) taken on a given day. However, it is to be understood that the dosage may be varied depending upon the requirement of the patients and the severity of the condition being treated etc.

In one embodiment, treatment lasts about 12 weeks. In another embodiment, treatment lasts about 24 weeks. In another embodiment, treatment lasts about 36 weeks. In another embodiment, treatment lasts about 48 weeks. In another embodiment, treatment lasts longer than about 48 weeks.

In one embodiment, a herbal composition of the present disclosure (e.g. MLC901 or MLC1501) may be administered to a subject with SCI in combination with other known SCI treatments, such as with one or more further active agents effective to control pain and/or muscle spasticity. The one or more further active agents may be administered at the same time (e.g. simultaneously) or at different times (e.g. sequentially) and over different periods of time, which may be separate from one another or overlapping. In one embodiment there may be a synergistic effect. The one or more further active agents may be administered by the same or different routes from the herbal composition of the present disclosure. The one or more further active agent utilized, and the appropriate administration route and dose level, will be known to those in the art or could be readily determined by one skilled in the art. Typically, as is well known in the medical art, dosage regimens may depend on various factors including the patient's size, body surface area, age, the particular compound to be administered, sex, time and route of administration, general health, and other drugs being administered concurrently. While individual needs vary, determination of optimal ranges of effective amounts of each component is within the skill of the art. Typically, the dosage of the one or more further active agents will be the same or similar to that administered when the agent is used without a herbal composition of the present disclosure. When a herbal composition of the present disclosure is administered with one or more further active agents, the one or more further active agents may be provided in a composition or kit comprising said herbal composition of the present disclosure, or the one or more further active agents may be provided separately (i.e. not as part of the composition or kit providing the herbal composition of the present disclosure).

In addition to the aforementioned embodiments, the present disclosure includes the following specific embodiments:
Embodiment 1: A herbal composition comprising at least the four herbal components Radix polygalae (root of thin leaf milkwort), Radix astragali (root of membranous milkvetch), Rhizome Ligusticum chuanxiong and Radix Angelica sinensis (root of Chinese angelica) or extracts thereof for use in treating a subject to induce and/or accelerate recovery from a spinal cord injury.
Embodiment 2: The herbal composition for use according to Embodiment 1, wherein the herbal composition further comprises at least one herbal component selected from the group consisting of Radix et Rhizome salviae Miltiorrhizae (red sage root), Radix paeoniae rubra (red peony root), flower of Carthamus tinctorius (safflower), Semen persicae (Prunus persica seed) and Rhizome Acori tatarinowii (rhizome of grassleaf sweetflag) or an extract thereof.
Embodiment 3: The herbal composition for use according to Embodiment 1, wherein the herbal composition consists essentially of at least the four herbal components Radix polygalae (root of thin leaf milkwort), Radix astragali (root of membranous milkvetch), Rhizome Ligusticum chuanxiong and Radix Angelica sinensis (root of Chinese angelica) or extracts thereof.
Embodiment 4: The herbal composition for use according to Embodiment 1, wherein the herbal composition consists essentially of at least the nine herbal components Radix polygalae (root of thin leaf milkwort), Radix astragali (root of membranous milkvetch), Rhizome Ligusticum chuanxiong, Radix Angelica sinensis (root of Chinese angelica), Radix et Rhizome salviae Miltiorrhizae (red sage root), Radix paeoniae rubra (red peony root), flower of Carthamus tinctorius (safflower), Semen persicae (Prunus persica seed) and Rhizome Acori tatarinowii (rhizome of grassleaf sweetflag) or extracts thereof.
Embodiment 5: The herbal composition for use according to any one of Embodiments 1 to 4, wherein extracts of each of the herbal components are used in the composition.
Embodiment 6: The herbal composition for use according to any one of Embodiments 1 to 5, wherein the herbal composition is a pharmaceutical composition that also comprises one or more pharmaceutically acceptable carriers or excipients.
Embodiment 7: The herbal composition for use according to Embodiment 6, wherein the pharmaceutical composition is MLC901.
Embodiment 8: The herbal composition for use according to Embodiment 6, wherein the pharmaceutical composition is MLC1501.
Embodiment 9: The herbal composition for use according to any one of Embodiments 1 to 8, wherein recovery from spinal cord injury is induced and/or accelerated by neuronal cell regeneration.
Embodiment 10: The herbal composition for use according to any one of Embodiments 1 to 9, said herbal composition promotes the growth of neuronal cells.
Embodiment 11: The herbal composition for use according to Embodiment 9 or Embodiment 10, wherein the neuronal cells are vertebrate neuronal cells.
Embodiment 12: The herbal composition for use according to Embodiment 11, wherein the vertebrate neuronal cells are human neuronal cells.
Embodiment 13: The herbal composition for use according to one of Embodiments 9 to 12, wherein the neuronal cells are derived from cortical neuronal cells.
Embodiment 14: The herbal composition for use according to any one of Embodiments 1 to 13, wherein administration of the herbal composition results in spinal cord regeneration. Embodiment 15: The herbal composition for use according to any one of Embodiments 1 to 14, wherein administration of the herbal composition results in regeneration of a connection between brain and spinal cord.
Embodiment 16: The herbal composition for use according to any one of Embodiments 1 to 15, wherein administration of the herbal composition results in regeneration of damaged neuronal tissue or cells.
Embodiment 17: The herbal composition for use according to any one of Embodiments 1 to 16, wherein administration of the herbal composition promotes limb motion recovery in paralyzed subjects.
Embodiment 18: The herbal composition for use according to any one of Embodiments 1 to 17, wherein an effective amount of the herbal composition is from about 1 mg/kg to about 100 mg/kg.
Embodiment 19: The herbal composition for use according to any one of Embodiments 1 to 18, comprising administering a second agent for treating a subject with spinal cord injury.
Embodiment 20: The herbal composition for use according to Embodiment 19, wherein the second agent is a pharmaceutical drug effective to control pain and/or muscle spasticity.
Embodiment 21: A herbal composition comprising at least the four herbal components Radix polygalae (root of thin leaf milkwort), Radix astragali (root of membranous milkvetch), Rhizome Ligusticum chuanxiong and Radix Angelica sinensis (root of Chinese angelica) or extracts thereof for use in the manufacture of a medicament for treating a subject to induce and/or accelerate recovery from a spinal cord injury.
Embodiment 22: The herbal composition for use according to Embodiment 21, wherein the herbal composition further comprises at least one herbal component selected from the group consisting of Radix et Rhizome salviae Miltiorrhizae (red sage root), Radix paeoniae rubra (red peony root), flower of Carthamus tinctorius (safflower), Semen persicae (Prunus persica seed) and Rhizome Acori tatarinowii (rhizome of grassleaf sweetflag) or an extract thereof.
Embodiment 23: The herbal composition for use according to Embodiment 21, wherein the herbal composition consists essentially of at least the four herbal components Radix polygalae (root of thin leaf milkwort), Radix astragali (root of membranous milkvetch), Rhizome Ligusticum chuanxiong and Radix Angelica sinensis (root of Chinese angelica) or extracts thereof.
Embodiment 24: The herbal composition for use according to Embodiment 21, wherein the herbal composition consists essentially of at least the nine herbal components Radix polygalae (root of thin leaf milkwort), Radix astragali (root of membranous milkvetch), Rhizome Ligusticum chuanxiong, Radix Angelica sinensis (root of Chinese angelica), Radix et Rhizome salviae Miltiorrhizae (red sage root), Radix paeoniae rubra (red peony root), flower of Carthamus tinctorius (safflower), Semen persicae (Prunus persica seed) and Rhizome Acori tatarinowii (rhizome of grassleaf sweetflag) or extracts thereof.
Embodiment 25: The herbal composition for use according to any one of Embodiments 21 to 24, wherein extracts of each of the herbal components are used in the composition.
Embodiment 26: The herbal composition for use according to any one of Embodiments 21 to 25, wherein the herbal composition is a pharmaceutical composition that also comprises one or more pharmaceutically acceptable carriers or excipients.
Embodiment 27: The herbal composition for use according to Embodiment 26, wherein the pharmaceutical composition is MLC901.
Embodiment 28: The herbal composition for use according to Embodiment 26, wherein the pharmaceutical composition is MLC1501.
Embodiment 29: The herbal composition for use according to any one of Embodiments 21 to 28, wherein recovery from spinal cord injury is induced and/or accelerated by neuronal cell regeneration.
Embodiment 30: The herbal composition for use according to any one of Embodiments 21 to 29, said herbal composition promotes the growth of neuronal cells.
Embodiment 31: The herbal composition for use according to Embodiment 29 or Embodiment 30, wherein the neuronal cells are vertebrate neuronal cells.
Embodiment 32: The herbal composition for use according to Embodiment 31, wherein the vertebrate neuronal cells are human neuronal cells.
Embodiment 33: The herbal composition for use according to one of Embodiments 29 to 32, wherein the neuronal cells are derived from cortical neuronal cells.
Embodiment 34: The herbal composition for use according to any one of Embodiments 21 to 33, wherein administration of the herbal composition results in spinal cord regeneration. Embodiment 35: The herbal composition for use according to any one of Embodiments 21 to 34, wherein administration of the herbal composition results in regeneration of a connection between brain and spinal cord.
Embodiment 36: The herbal composition for use according to any one of Embodiments 21 to 35, wherein administration of the herbal composition results in regeneration of damaged neuronal tissue or cells.
Embodiment 37: The herbal composition for use according to any one of Embodiments 21 to 36, wherein administration of the herbal composition promotes limb motion recovery in paralyzed subjects.
Embodiment 38: The herbal composition for use according to any one of Embodiments 21 to 37, wherein an effective amount of the herbal composition is from about 1 mg/kg to about 100 mg/kg.
Embodiment 39: The herbal composition for use according to any one of Embodiments 21 to 38, wherein the method comprises administering a second agent used for treating a subject with spinal cord injury.
Embodiment 40: The herbal composition for use according to Embodiment 39, wherein the second agent is a pharmaceutical drug effective to control pain and/or muscle spasticity.

In one further embodiment, the present disclosure provides an in vitro injury model for SCI using a mechanical or chemical method to inflict injury in NSC 34 cell lines.

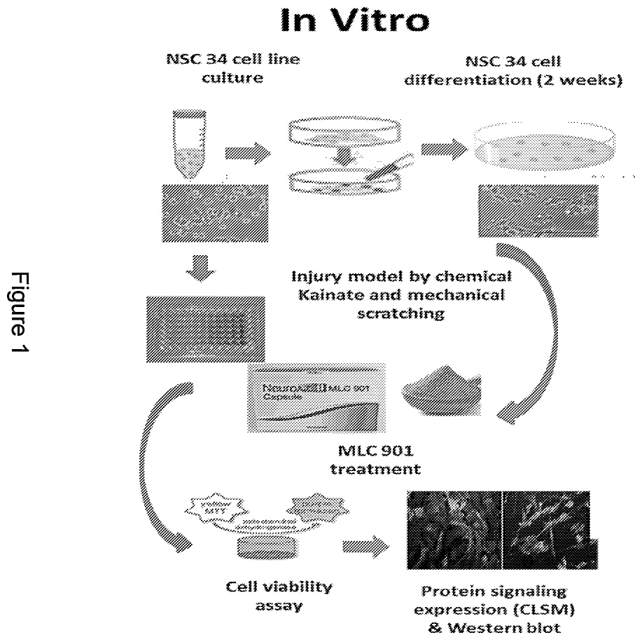

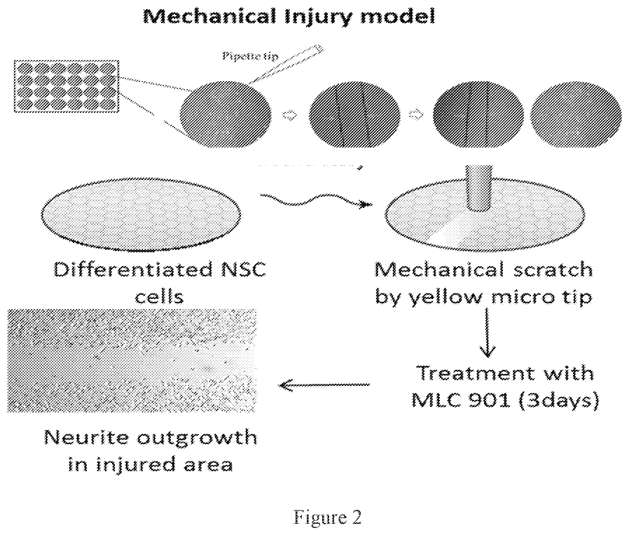

In one further embodiment, the in vitro mechanical injury method is shown in FIGS. 1 and 2.

In one further embodiment, the present disclosure has the following objectives:

1. Investigate the neurotoxicity effect of compositions of the present disclosure on NSC-34 cells.

2. Create an in vitro SCI injury model using NSC-34 cell line cultures.

3. Determine the effect of compositions of the present disclosure on the regeneration of neurites after injury in the in vitro SCI injury model using time-lapse imaging and immunofluorescence labeling.

4. Study the underlying protein signaling pathway during neuronal differentiation and regeneration when supplemented with compositions of the present disclosure.

### In Vitro Mechanical Injury Model

### Objective 1: Investigate the Neurotoxicity Effect of Compositions of the Present Disclosure on NSC-34 Cells

Method: NSC-34 cell lines were cultured in DMEM/F12 (1:1)+10% FBS and 1% AA, and allow to grow until 90% confluency, then treated with differentiation media consisting of DMEM/F12 (1:1)+1% FBS+1% NEAA+1% AA and 1 μM RA, 10 μM RA or 30 μM PGE2 and allowed to differentiate into mature motor neurons. The differentiation was determined by estimating neurite outgrowth from Day 0 to Day 14 by ImageJ and ICC staining using differentiation marker beta III tubulin and DAPI. The differentiated motor neurons were seeded at a rate of 5000 cells/well into a 96 well plate and allowed to attach for 24 h. Different concentrations (ranging from 25 μg/mL-2000 μg/mL) of MLC901 were then added to the wells and incubated for 24 and 48 hours. The neurotoxicity of MLC901 was estimated using an MTT salt assay and plate reader at 595 nm wavelength.

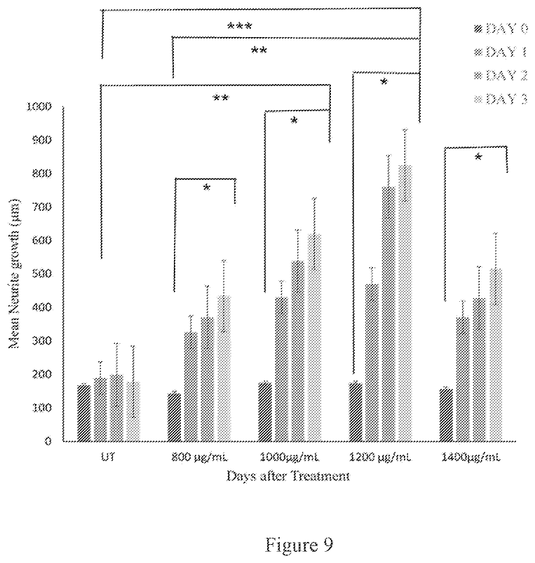

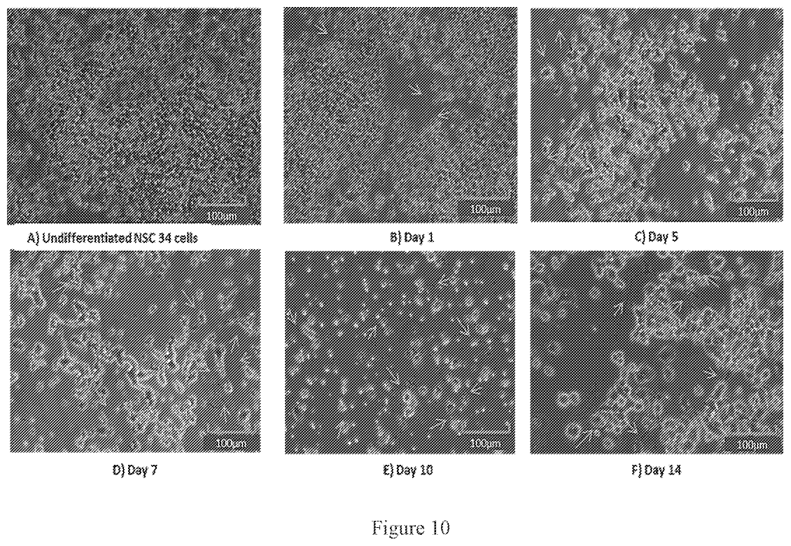

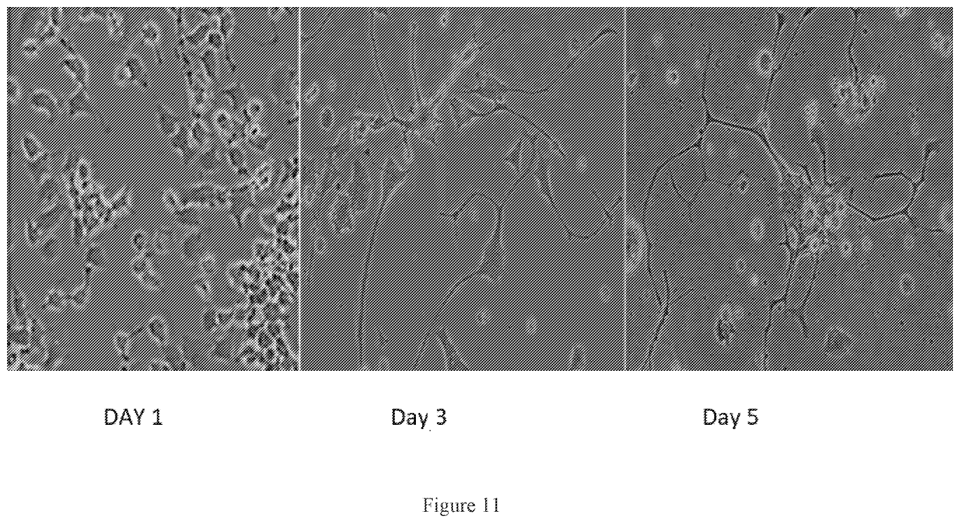

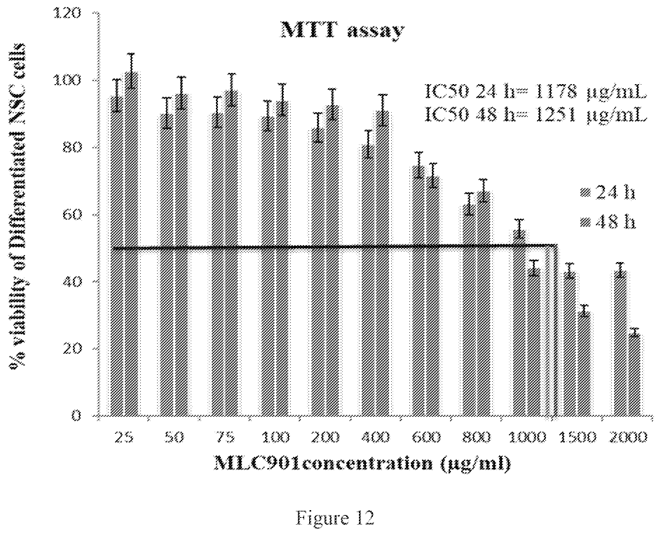

Result: The NSC-34 cell differentiation shown in FIG. 10 reflects mature motor neuron at Day 10 and Day 14 of differentiation using 1 μM RA, 10× magnification. The NSC-34 cell differentiation shown in FIG. 11 reflects mature motor neurons at Day 3 and Day 5 of differentiation using 30 μM PGE2, 10× magnification. FIG. 12 shows a bar graph reflecting percentage viability of differentiated NSC-34 cells to different MLC901 concentrations. The MLC901 neurotoxicity results show the percentage viability of differentiated NSC-34 cells to MLC901 at concentrations ranging from 25 μg/ml to 2 mg/ml by MTT assay, where IC 50 values at 24 hours and 48 hours are 1178 μg/ml and 1251 μg/ml respectively (n=6; results shown as mean±SD). FIG. 13 shows an NSC-34 cell differentiation study on the 7th day The study used beta III tubulin and DAPI at 1 μM RA, 10 μM RA and 30 μM PGE2. 10 μM RA and 30 μM PGE2 showed more cells with longer neurite, (n=3).

Discussion: The neurotoxicity study using MLC901 gave an IC 50 value of differentiated NSC 34 cells of 1251 μg/mL. The treatment concentration range of 800 μg/mL to 1400 μg/mL was therefore selected for further studies. In the differentiation study using different differentiation conditions—1 μM or 10 μM RA and 30 μM PGE2—it was found that 1 μM RA gave NSC-34 cell differentiation in 14 days, 10 μM RA gave NSC-34 cell differentiation in 7 days, and 30 μM PGE2 gave NSC-34 cell differentiation in 3 to 5 days. Therefore 30 μM PGE2 was used for differentiation in the in vitro mechanical injury studies.

### Objective 2: Create an In Vitro Spinal Cord Injury Model Using NSC-34 Cell Line Cultures

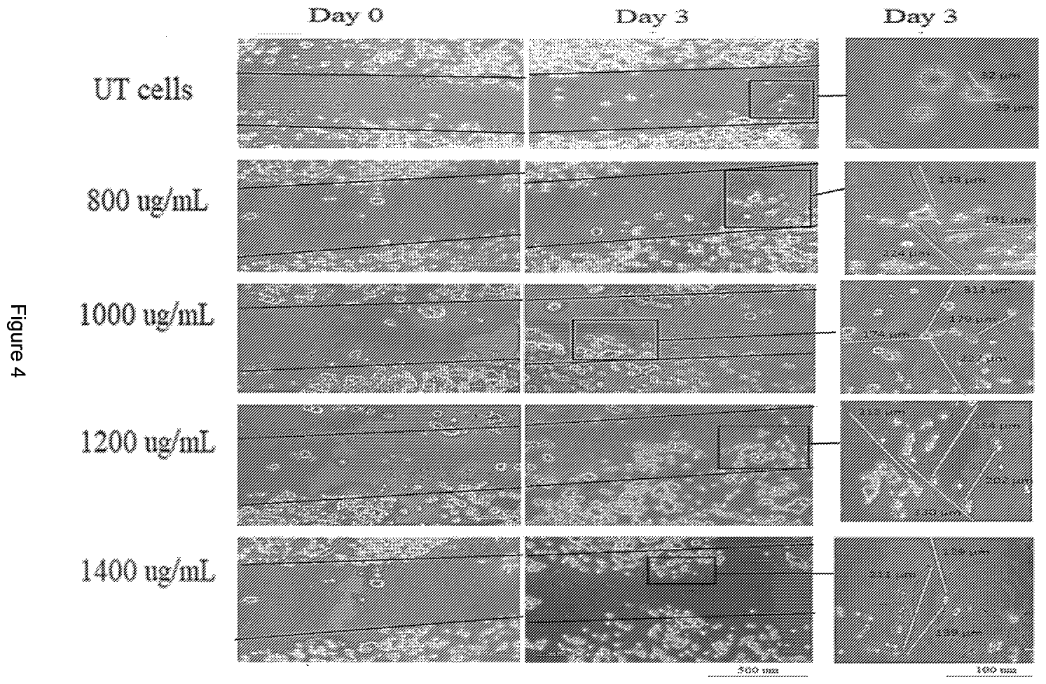

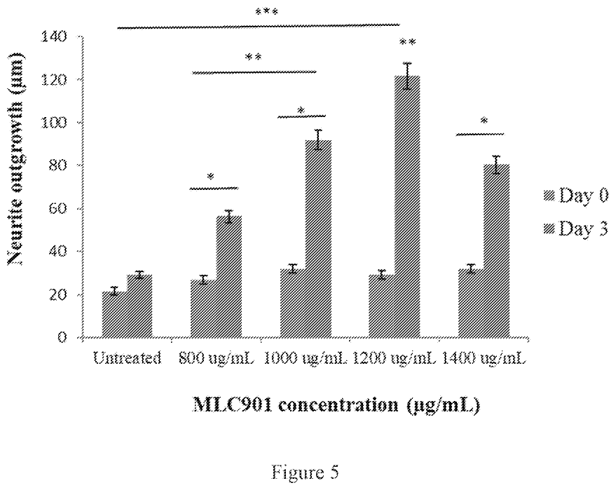

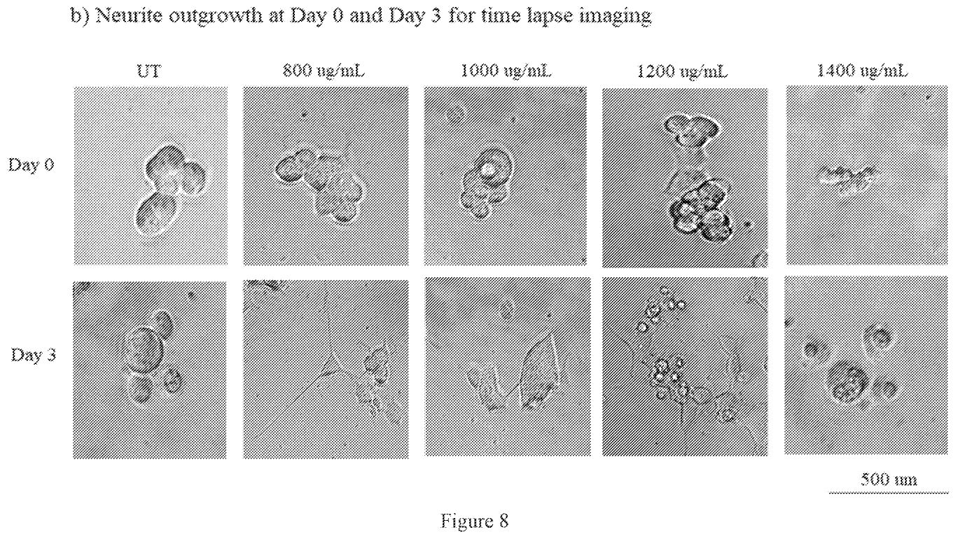

Method: Mechanical scratching of the NSC-34 cell culture was carried out using a pipette tip, making two parallel vertical scratches. The cells were then washed with DPBS three times and observed under a microscope to examine the damaged neuronal cells at the scratch area. The injured cells were treated with MLC901 at concentrations of 800, 1000, 1200 and 1400 μg/mL for 3 days and neurite regeneration was estimated from neurite outgrowth and neurite count measurements at the area of injury by ImageJ, time lapse assay and ICC staining. FIG. 5 shows a bar graph of the neurite outgrowth at different MLC901 concentrations at Day 0 and Day 3. FIG. 6 shows a bar graph of the neurite counts at different MLC901 concentrations at Day 0 and Day 3. In these experiments, concentrations of MLC901 of 1000 μg/mL and 1200 μg/mL showed longer neurite (p>0.05) outgrowth than concentrations of 800 μg/mL and 1400 μg/mL (n=6). FIG. 9 shows a bar graph of neurite growth after time lapse imaging at Day 0, Day 1, Day 2 and Day 3 using different MLC901 concentrations. The neurite outgrowth is shown for untreated cells and cells treated with MLC901 at concentrations of 800 μg/mL, 1000 μg/mL, 1200 μg/mL and 1400 μg/mL by ImageJ software. All treatment groups showed longer neurite (p>0.05) outgrowth on Day 3 as compared to Day 0, 800 and 1000 μg/mL showed significant (p<0.01) neurite outgrowth as compared to UT cells and cells treated at 1200 μg/mL (p<0.001; n=6). In FIGS. 5, 6 and 9 * indicates p<0.05, ** indicates p<0.01 and *** indicates p<0.01.

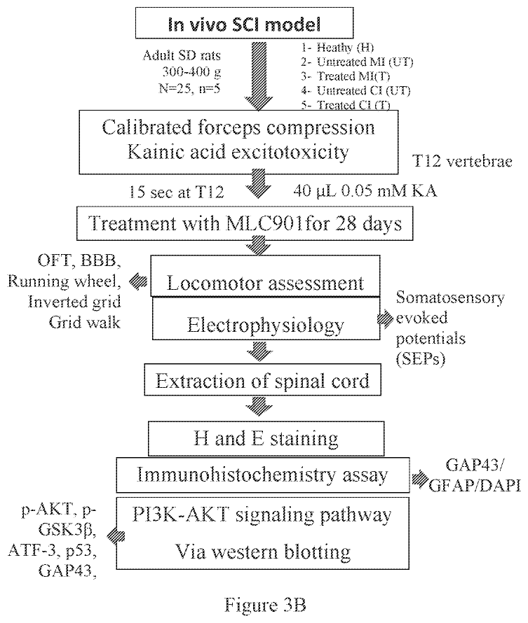

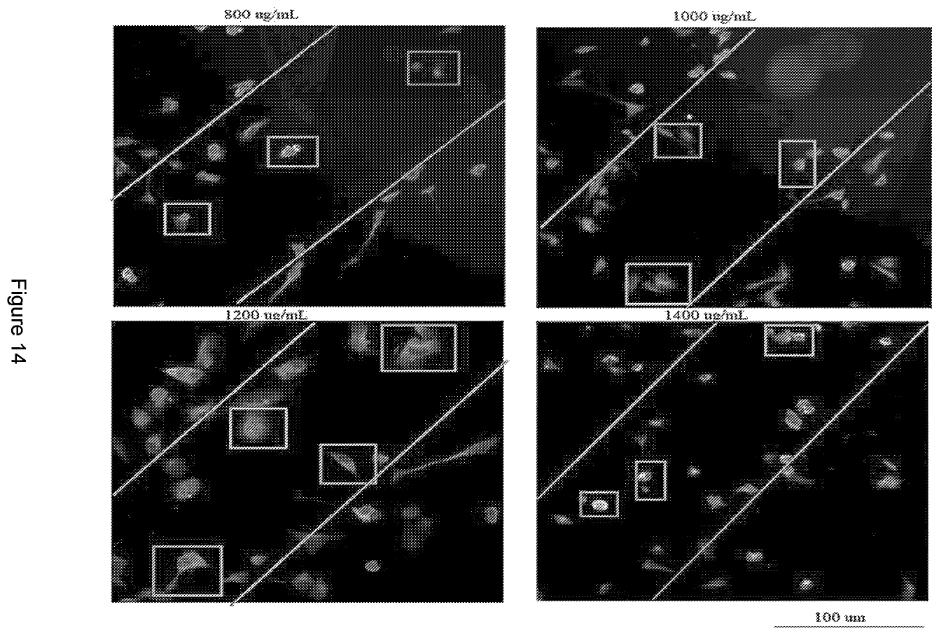

Results: FIG. 4 shows a mechanical scratch injury model at Day 0 and Day 3 following treatment with different concentrations of MLC901, i.e. a) control cells with media only, b) 800 μg/mL MLC901, c) 1000 μg/mL MLC901, d) 1200 μg/mL MLC901 and e) 1400 μg/mL MLC901. The lines drawn in FIG. 4 indicate the area of mechanical injury and arrows show neurite outgrowth, including magnification 10× of the selected area and a scale bar of 100 μm. Day 3 of treatment showed a higher neurite outgrowth as compared to the untreated group, with the MLC901 concentrations of 1000 and 1200 μg/mL providing the highest neurite outgrowth at the injured area (n=6). FIG. 15 shows a bar graph reflecting percentage viability of cells to different MLC901 concentrations as compared to untreated cells. Here, MLC administered at 1200 μg/mL showed superior cell viability (p>0.05) compared to untreated cells, and cells treated with MLC901 at 800 μg/mL and 1400 μg/mL (n=3).

Discussion: These results indicate that MLC901 treatment promotes neurite outgrowth at the injured area especially at concentrations of 1000 μg/mL and 1200 μg/mL. It was also observed that the neurite outgrowth in treatment groups was better than in the untreated group. Therefore, the results clearly indicate that MLC901 promotes neurite outgrowth in both healthy and injured cells culture of differentiated NSC 34 cells. MTT assay shows the cells with injury having MLC901 treatment group show better percentage viability as compared to the untreated group at the injured area with treatment and without treatment.

Objective 3: Determine the Effect of Compositions of the Present Disclosure on the Regeneration of Neurites after Injury in the In Vitro SCI Injury Model Using Time-Lapse Imaging and Immunofluorescence Labeling

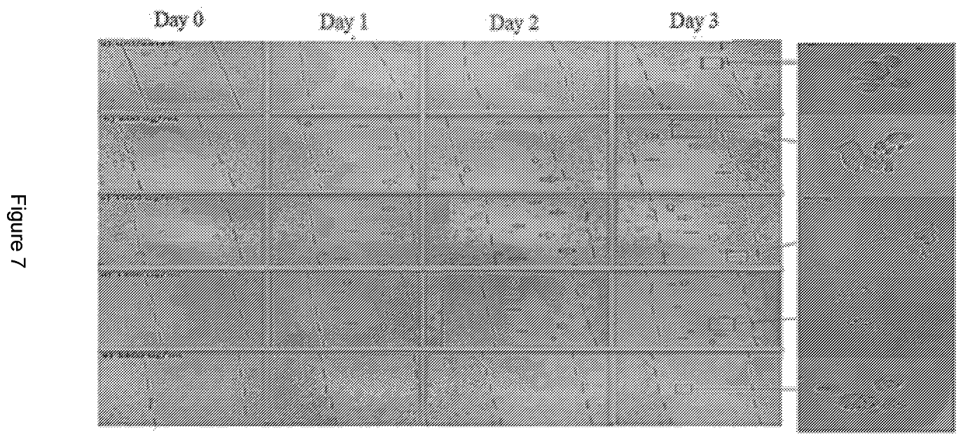

Method: Mechanical scratching of NSC-34 cell culture was carried out using the procedure described above, and the cells were then treated with different MLC901 concentrations (i.e. 800 μg/mL 1000 μg/mL, 1200 μg/mL and 1400 μg/mL), prepared in media and placed in the incubator at 37° C. Neurite regeneration was observed over 72 hours using a camera with pictures captured every 30 minutes. The regenerative ability of MLC901 at the injured area was compared to untreated cells at different points of the observation period.. After 3 days of treatment and time lapse imaging, the cells, as shown in FIG. 8, were stained with beta III tubulin and DAPI to further observe neuro-regeneration. The lengths of neurite outgrowth were measured and statistically analyzed at 20× magnification.

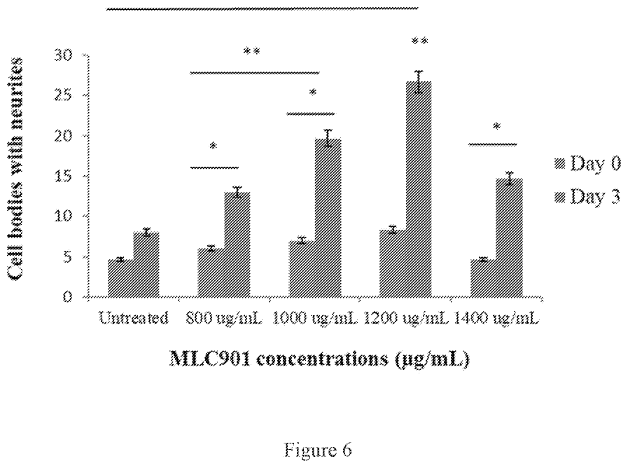

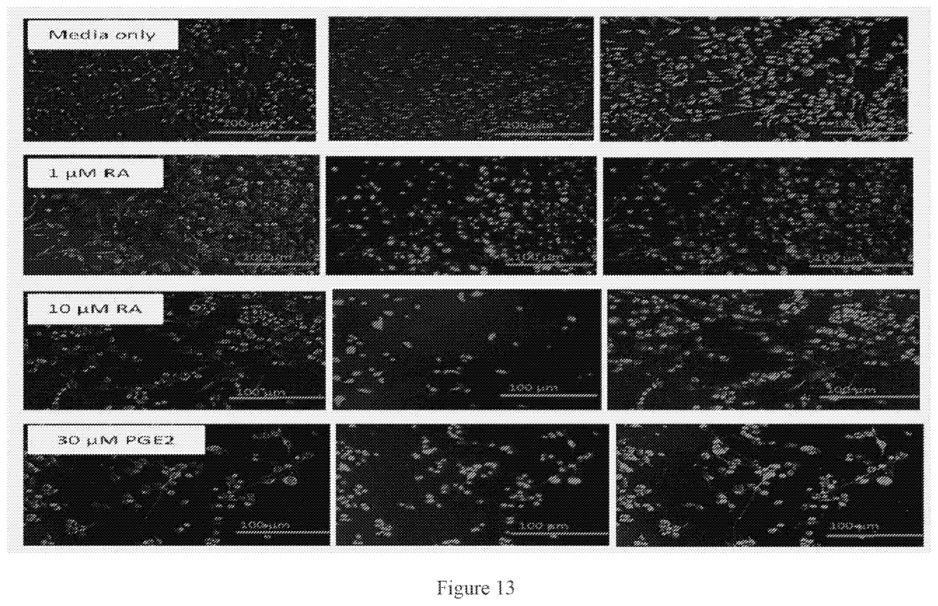

Result: FIG. 7 shows time lapse imaging demonstrating that MLC901 promotes neurite outgrowth and neurite count at the injured area, with 1000 μg/mL and 1200 μg/mL showing greater neurite outgrowth at the injured area, (n=3) than the other concentrations. The neurite outgrowth was quantified by using ImageJ software. FIG. 8 shows time lapse imaging of neurite outgrowth of injured NSC34 cells on Day 0 and Day 3. FIG. 14 shows ICC staining using DAPI and beta III tubulin on Day 3 following treatment of NSC-34 cells with different concentrations of MLC901 (i.e. 800 μg/mL 1000 μg/mL, 1200 μg/mL and 1400 μg/mL). The lines drawn in FIG. 4 indicate the area of mechanical injury and arrows show neurite outgrowth, including magnification 10× of the selected area and a scale bar of 100 μm. Day 3 of treatment showed a higher neurite outgrowth as compared to the untreated group, with the MLC901 concentrations of 1000 and 1200 μg/mL providing the highest neurite outgrowth at the injured area (n=6).

Discussion: Time lapse imaging shows that MLC901 promotes neurite outgrowth compared to untreated cells, with more longer neurite growth at the injured area when MLC901 is administered at concentrations of 1000 μg/mL and 1200 μg/mL. ICC staining after time lapse imaging also demonstrated more expression of beta III tubulin following administration of MLC901 at concentrations of 1000 μg/mL and 1200 μg/mL, indicating that these two concentrations were the best for neurite regeneration.

Objective 4: Study the Underlying Protein Signaling Pathway During Neuronal Differentiation and Regeneration when Supplemented with Compositions of the Present Disclosure

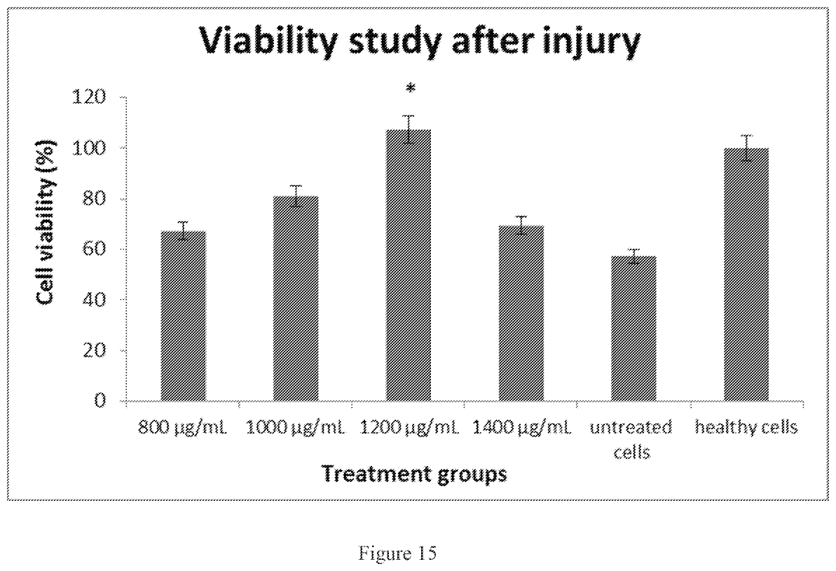

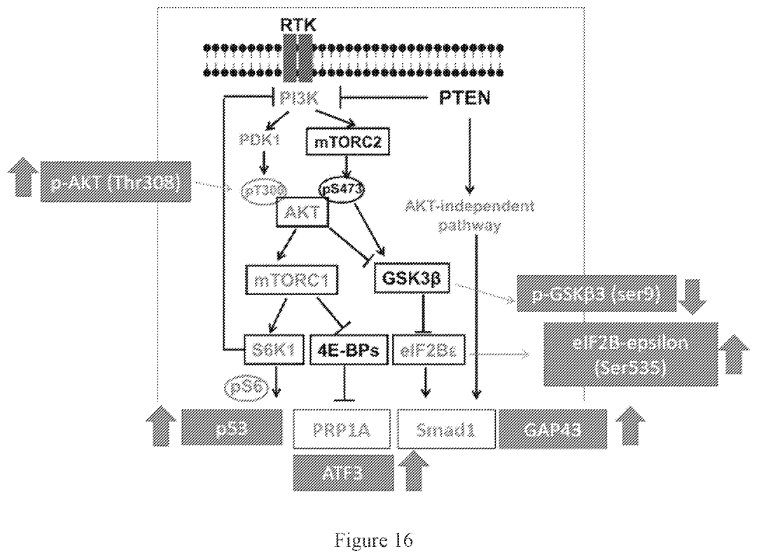

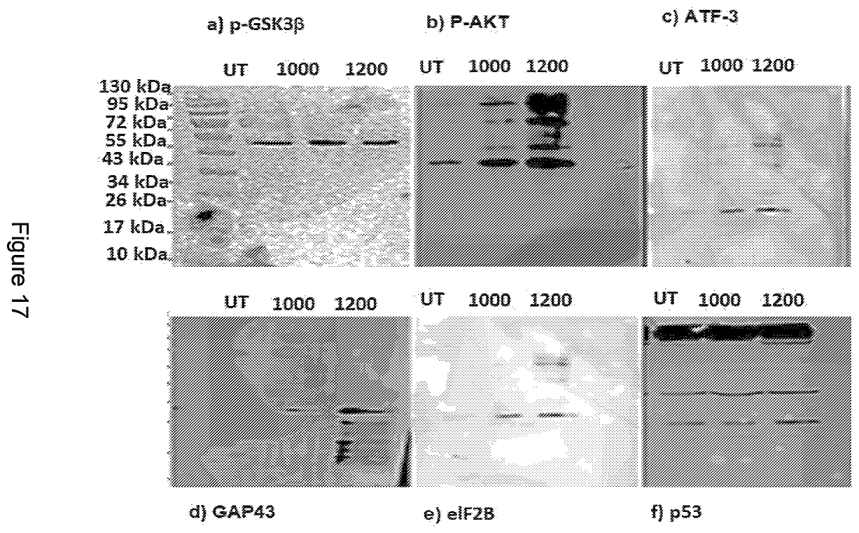

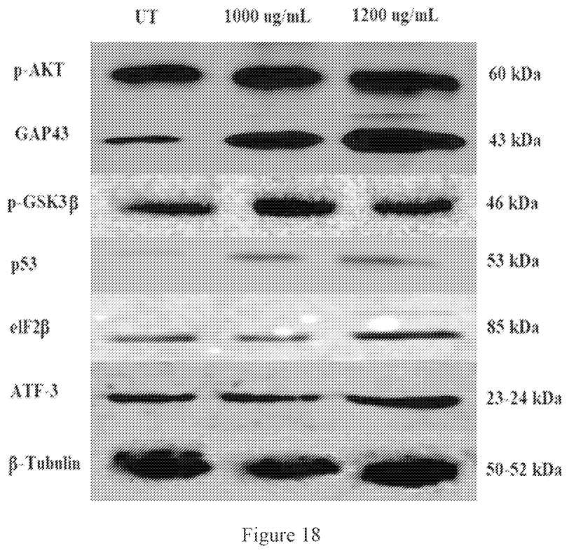

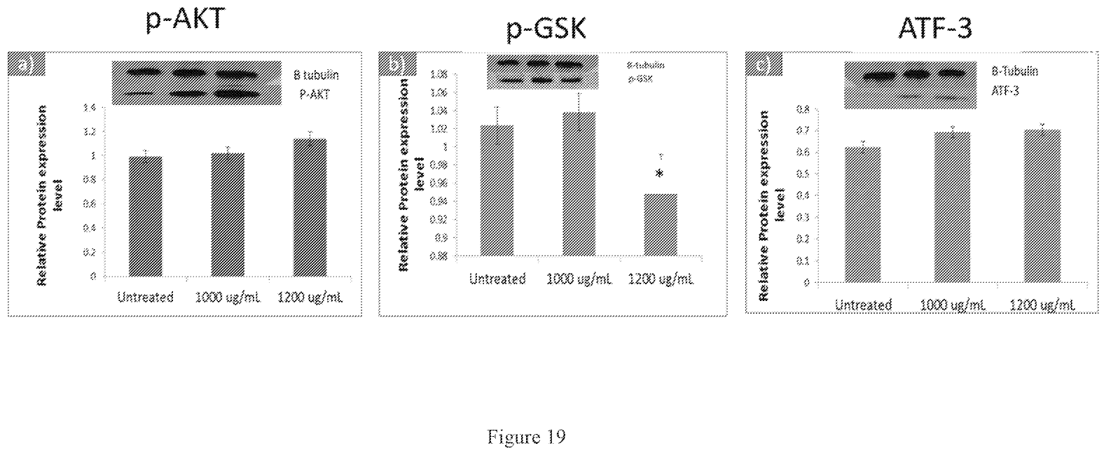

Method and results: The phosphoinositide 3-kinase (PI3K)/AKT signaling pathway, as shown in FIG. 16, contributes to a variety of processes, mediating many aspects of cellular functions, including nutrient uptake, anabolic reactions, cell growth, proliferation, and survival. Expression of different markers for this pathway indicate neuro-regeneration. For example, up-regulation of p-AKT(Thr308) indicates regeneration. The down-regulation of p-GSKβ3(Ser9) reflects regeneration. Similarly, elevated expression of p53, ATF3, GAP43 and eIF2B(ser535) indicate neuronal regeneration. FIG. 17 shows a Western blot analysis of the expression of the protein markers phospho-AKT(Thr308), phospho-GSKβ3(Ser9), ATF-3, GAP43, eIF2β (ser535) and p-53 in untreated (UT) injured NSC34 cells, and injured NSC34 cells treated with MLC901 at concentrations of 1000 μg/mL and 1200 μg/mL. 1200 μg/mL MLC901 produced more protein marker bands than 1000 μg/mL MLC901, with 1200 μg/mL MLC901 showing less expression of phospho-GSKβ3(Ser9) than 1000 μg/mL MLC901, indicating than 1200 μg/mL MLC901 provides superior neuro-regeneration. FIG. 18 shows a Western blot analysis of the expression of proteins involved in the PI3K/AKT neuro-regeneration pathways in injured NSC34 cells. Cells examined consisted of untreated cells and cells treated with MLC901 at concentrations of 1000 μg/mL and 1200 μg/mL. The cells treated with MLC901 at a concentration of 1200 μg/mL showed the highest expression of neuro-regenerative proteins. FIG. 19 is a bar graph reflecting the expression of PI3K/AKT pathway proteins a) phospho-Akt(Thr308), b) phospho-GSK3β(Ser9), and c) ATF-3 in different MLC901 concentration groups (namely untreated control, 1000 μg/mL, and 1200 μg/mL) for 72 h. Phospho-Akt(Thr308) and ATF-3 showed up-regulation while phospho-GSK3β(Ser9) showed statistically significant down-regulation compared to the untreated cells indicating neuro-regeneration. FIG. 20 is a bar graph reflecting the expression of PI3K/AKT pathway proteins d) GAP-43, e) p53, and f) eIF2β(ser535) in the different MLC901 concentration groups (namely untreated control, 1000 μg/mL, and 1200 μg/mL) for 72 h. eIF2β (ser535) showed statistically significant (p>0.01) up-regulation at 1000 μg/mL and 1200 μg/mL as compared to the untreated cells, indicating regeneration.

Discussion: The higher expression of markers in 1200 μg/mL MLC901 indicates more neurite regeneration as compared to the untreated cells. Similarly, less expression of phospho-GSK3β(Ser9) at 1200 μg/mL also indicates neuronal regeneration. These results provide support for use of MLC901 as a neuro-regenerative drug to treat SCI.

In one further embodiment, the present disclosure provides an in vivo injury model for SCI using mechanical and chemical methods in rats.

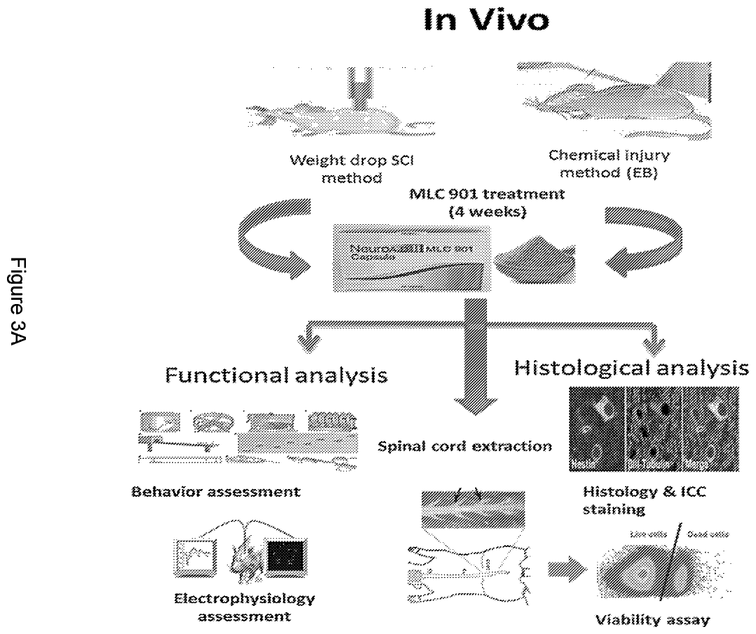

In one further embodiment, the in vivo SCI injury method is shown in FIG. 3a or 3b.

In one further embodiment, the present disclosure has the following objectives:

1. Establish an in vivo mechanical SCI injury model in adult rats.

2. Investigate the efficacy of compositions of the present disclosure on neuronal regeneration in rats whose spinal cords are injured by mechanical means (e.g. compression).

3. Establish an in vivo chemical SCI injury model in adult rats.

4. Investigate the efficacy of compositions of the present disclosure on neuronal regeneration in rats whose spinal cords are injured by chemical means (e.g. using kainic acid).

In vivo spinal cord injury (SCI) models are considered non-replaceable as they can relate to similar pathophysiological conditions in humans. SCI models aid understanding of the injury mechanisms and benefit analysis of advanced therapeutic interventions. Due to their close analogy in the functional, morphological, and electrophysiological consequences of SCI in humans, the most commonly utilised animals for studying various neuronal pathological conditions are rodents, including rats and mice.

SCI is classified either as complete or incomplete injury. A complete SCI describes the complete loss of sensation and muscle function at and below the injury site. An incomplete SCI refers to partial function loss below the injury level. The level of injury is another crucial aspect: SCI in the cervical and upper thoracic region can cause inconsistent breathing patterns and lead to death. Injury in the lower thoracic or lumbar region is preferable as an SCI model as it only produces paraplegia without altering respiratory and cardiac functions. SCI models are categorised based on the mechanism of injury: mechanical or chemical. Mechanical injury is caused by mechanical means such as impactors, forceps, clips, balloons, or scissors, while chemical injury is caused by injecting chemicals such as glutamate, aspartate, N-methyl-D-aspartate (NMDA), superoxide, hydroxyl radical and peroxy-nitrate, heavy metals, ethidium bromide, or kainate.

There are various mechanical injury models for producing complete and incomplete SCI, such as contusion, compression, distraction, dislocation, or transection. The mechanical injury model is advantageous for assessing axonal regeneration and subsequent functional recovery. The chemical injury model is useful for investigating axonal and neuronal degeneration, molecular mechanisms, and the effect of various therapies on specific pathways. The excitotoxic chemical injury model is gaining popularity as it is useful for studying secondary injury mechanism events such as neuronal and axonal degeneration caused by glutamate excitotoxicity. Neurodegeneration is described as the progressive loss of structure and function of neurons, axons, and nerve cells. Chronic neurodegenerative diseases, such as Parkinson disease, Huntington disease, Alzheimer's disease, temporal lobe epilepsy, and amyotrophic lateral sclerosis (ALS) occur because of chemical excitotoxicity. 6-Hydroxydopamine (6-OHDA) causes neurotoxicity that produces Parkinson disease and G93A mutation causes the hydroxyl radical production in transgenic ALS rats, which further implicates oxidative damage causing ALS pathogenesis. The exposure of the brain and spinal cord to other chemicals, such as heavy metals (e.g. aluminium) cause cognitive impairment and generally damage the nervous system, while scopolamine causes dementia, colchicine induces Alzheimer's disease symptoms, and kainic acid (KA) produces temporal lope epilepsy via intrahippocampal or intra-amygdaloid administration or SCI via intra-spinal administration.

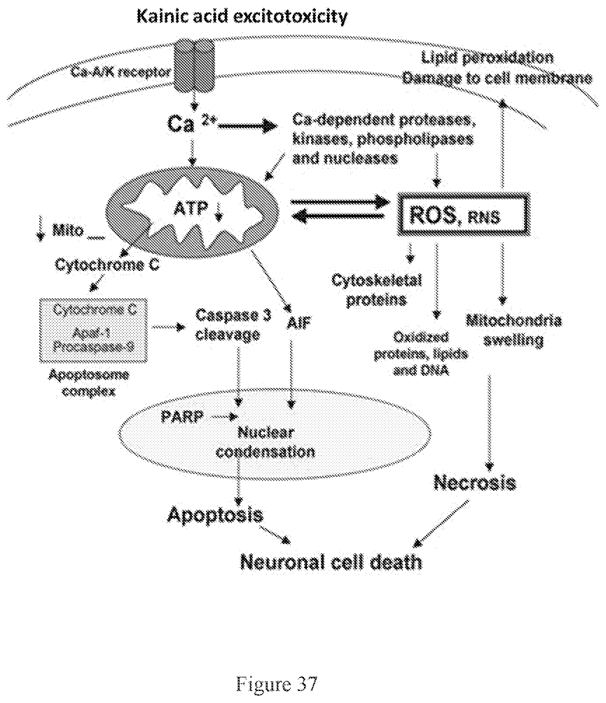

Kainic acid (KA) is an agonist for ionotropic glutamate receptors, which induces neuropathological changes both in vivo and in vitro, and is commonly used to study the mechanism of excitation-induced neuronal apoptosis. Excitotoxicity has a fundamental role in many nervous system disorders, including brain and spinal ischemia, trauma, and other neurodegenerative disorders. L-glutamate is the major excitatory transmitter located in the nervous system. It acts as a synaptic neurotransmitter, inducing long-lasting changes in synaptic organization, neuronal migration, neuronal excitability during development stages, and ensuring neuronal viability. The overactivation of glutamate receptors by KA alleviates intra-cellular calcium ion influx, dominating the production of free radicals, i.e. ROS (reactive oxygen species) and RNS (reactive nitrogen species), along with ATPase, which detonate additional influx of harmful ions and chemicals creating neuronal death. Administration of KA provokes glutamate mediated excitotoxicity, leading to neuronal death and neurodegeneration. The proposed mechanism of action of KA-induced excitotoxicity is depicted in FIG. 37.

The utilization of KA-induced neuronal excitotoxicity models is a helpful way to screen potential therapeutic drugs for nerve regeneration in SCI. A model of KA-induced SCI in Sprague Dawley (SD) rats is described hereinafter. In this study, the locomotor, electrophysiological, neurological and histological changes occurring after intra-spinal administration of KA followed by treatment with MLC 901 were measured to assess the ability of MLC901 to induce nerve regeneration in SCI.

The test used in the in vivo studies are as follows:

### Locomotor Activity

### (a) Open Field Test

The Open Field Test (OFT) was conducted to evaluate changes in the locomotor activity of the rats. The rats were placed in the middle part of an acrylic box and each rat's locomotor activity was observed for 5 minutes by two blinded observers. The rats were then scored based on the BBB scale. The floor of the BBB scale was divided into three different parts. The inner square comprised of 20 cm, the middle square comprised of 40 cm and the outer square composed of 60 cm distance. The floor of the Open Field was marked with 10 cm×10 cm boxes, and the number of boxes crossed during the 5-minute assessment by all groups at days 3, 7, 14, 21 and 28 following injury were recorded and compared.

### (b) Running Wheel Test

Wheel running is one of the most widely used tests to evaluate motor deficits in rodents having brain and spinal cord injuries. In this test a rat was placed into the wheel and then the wheel was rotated forcefully at 90°. The rat tried to restrain the wheel movement by gripping it using forelimb and hind limb coordination, and the time taken for the rat to completely restrain and stop the wheel was recorded for each rat in all groups. The scale used is described below (0-4).

| | |
|---|---|
| 0 | No restraining |
| 1 | Poor restraining |
| 2 | Slight restraining |
| 3 | Moderate Restraining |
| 4 | Complete restraining |

### (c) Grid Walk Test

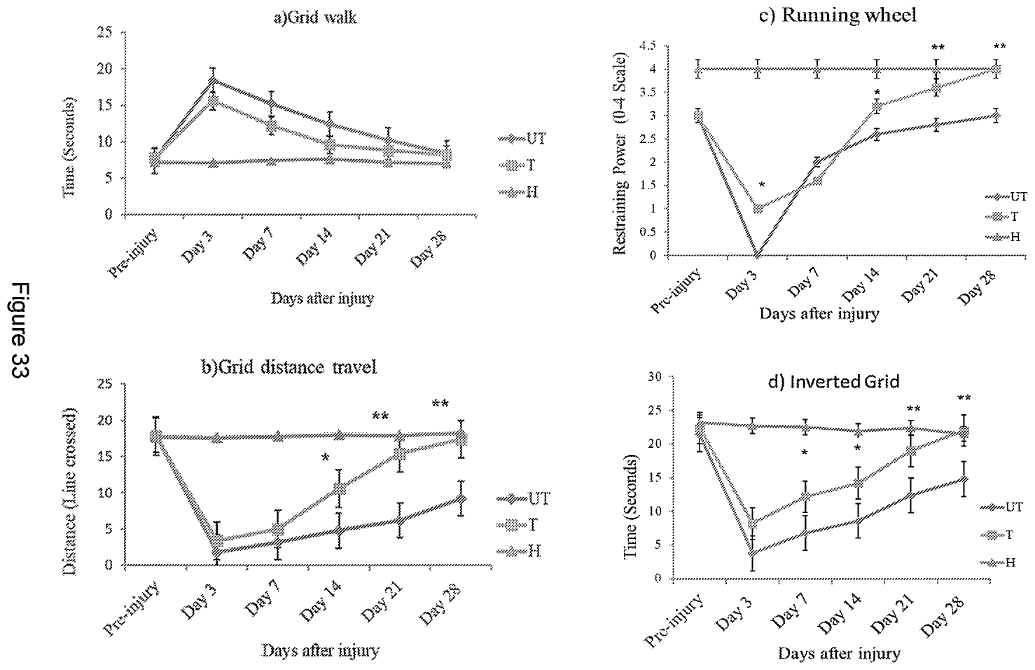

An elevated metal square grid (40×60 cm) was used in this test [see FIG. 33c)]. The grid apparatus was placed in the Open Field apparatus described above. Rats were placed at one end of the grid and allowed to walk and reach the other end of grid. Walking behaviors, such as the number of faults, total footsteps and time taken to walk the grid were recorded and were then analyzed by an experimenter who was blind to the experimental design.

### (d) Inverted Grid Test

The inverted grid test is a test to estimate muscle strength for forelimbs and hindlimbs. Usually, rats can easily hold onto the grid for 30 to 40 seconds in inverted position. Hence, to evaluate the strength after injury, the rats were placed upside down on a grid approximately 20 cm above the ground and a timer was set. The time each rat was able to hold onto the gird in a inverted position was recorded.

### Sensory Function Tests

### (a) Hot Spatula Method

The hot spatula test was used to evaluate the changes in sensory nociception of the rats. A standard temperature was determined by placing a hot spatula on the tail of a control rat with the temperature being raised slowly and stopped when the rat started to lick its tail. The mean temperature calculated for all the control rats was used as the standard temperature. The latency time for the sensation of temperature (tail licking) was noted and compared group-wise. An increase in latency time indicated loss of sensory nociception, with an increased threshold for a response to increasing temperature. The duration of stimulation was no longer than 20 seconds to avoid burn damage. The process was repeated three to five times, and the mean values were taken as the threshold values. The baseline for normal rats is around 12 seconds. A scale of 0-3 based on rat's behavior was used as described below:

| | |
|---|---|
| 0 | No withdrawal |
| 1 | Slow withdrawal |
| 2 | Prolonged withdrawal |
| 3 | Quick withdrawal and repeated flicking |

### (b) Cold Sensation Test

The cold sensation test is also useful for sensory function testing. Absolute alcohol (98%) is mainly used for testing the cold sensation of rats. First, the area of interest of the skin of a rat was shaved. Then absolute alcohol was sprayed onto the shaved area, and the response was recorded and graded based on the same scale described just above.

### Electro-Physiological Assessment

### Somatosensory Evoked Potentials (SEPs) Measurements

SEPs are brain and spinal cord responses elicited by motor and electrical stimulation to the peripheral nerve. The commonly used sites of stimulation in rats are the sciatic nerve at the wrist and posterior tibial nerve at the ankle. SEPs were measured in all rats in each group using Nicolet® Viking Quest™. Rats were anesthetized and then a stimulus electrode was fixed to the hind legs. Recording electrodes were placed on the hind-limb cortical sensory area between the coronal suture and sagittal suture lines. The reference electrodes were placed 0.5 cm posterior to the recording electrodes, eliciting direct-current square wave electrical stimulation with an intensity of 10-30 mA, pulse width of 0.1 ms, and frequency of 1 Hz, which was superimposed 50-60 times. SEP latency, duration and amplitude were recorded, and nerve electrophysiological recovery was observed in all groups.

Histological Analysis after Spinal Cord Extraction

After 4 weeks post-surgery and treatment, the rats were sacrificed. Spinal cord tissue (T12-T13) was dissected and fixed overnight in 4% paraformaldehyde, followed by 30% sucrose, and then cut into 15-mm thick sagittal and parasagittal sections using a cryostat. Hematoxylin-eosin (H&E) staining was performed for general histological examination under microscope observation.

### Immunohistochemical Analysis

Immunohistochemistry was carried on 5 μm thin slices of spinal cord tissue using specific antibodies. The sections were incubated in monoclonal GAP43 (D9C8) Rabbit antibody (1:200 cell signalling, USA) and GFAP Monoclonal Antibody (1:200 ThermoFisher Scientific) at 4° C. overnight in a humidified chamber, followed by staining with anti-rabbit IgG 488 and 594 secondary antibody (1:400; Santa Cruz Biotechnology, Santa Cruz, CA, USA) at 37° C. for 2 h. The nuclear staining was carried out by using DAPI (1;15000) in DPBS solution and incubated for 30 to 40 minutes at room temperature in the dark. Finally, the slides were mounted and viewed under confocal microscope (Nikon AiR).

### In Vivo Injury Models

### Objective 1: Establish an In Vivo Mechanical Spinal Cord Injury Model in Adult Rats (Calibrated Forceps Compression Method)

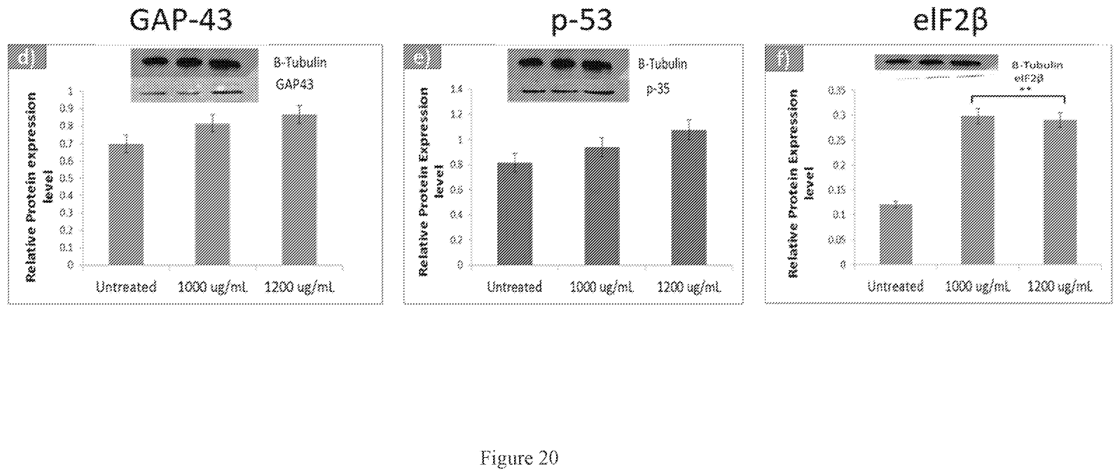

Method: The Calibrated Forceps Compression method was performed by using forceps (Dumont #5) to produce compression for 15 seconds at the T12 vertebrae. The arms of the forceps were placed at adjacent sides of the exposed spinal cord and compression applied until the bottom of the forceps tip touched each other. The forceps were held for 15 seconds at this position creating a moderate injury. FIG. 21 shows the compression method, where a) to h) represent the following: a) sublime animal position, b) marking T10, T12 and T13 vertebrae, c) subcutaneous cut, d) removing muscles, e) exposing spinal cord, f) removing T12 vertebrae, g) compression of spinal cord for 15 seconds and h) wound closing (suture of tissue and skin).

Objective 2: Investigate the Efficacy of Compositions of the Present Disclosure on Neuronal Regeneration in Rats Whose Spinal Cords are Injured by Mechanical Means (e.g. Compression)

Method: 30 minutes after spinal injury is mechanically induced in rats, MLC901 was first administered subcutaneously using a single solution dose of 10 mg/kg of MLC901 in normal saline, and then administered orally at 10 mg/kg/day in drinking water for 4 weeks. If the animal would not take the drug orally, then an oral gavage (20-16 G, 3.8 to 10 cm) technique was used. The oral gavage needle was gently advanced along the upper palate until the esophagus was reached. The tube should pass easily into the esophagus. Once proper placement was verified, the drug was slowly administered by a syringe attached to the end of the needle. After dosing, the needle was gently removed following the same angle as insertion. During treatment, the locomotor function was assessed using the Open Field Locomotor Scale (BBB). Other tests include running wheel, grid walk and inverted grid tests as discussed above. After treatment, rats were sacrificed (after 28 days) and their spinal cords extracted for further assessment. Post-sacrifice assessments may include electrophysiology (SEP and/or MEP), histology and immunohistochemistry, and PI3K/Akt pathway neurite regeneration protein marker tests.

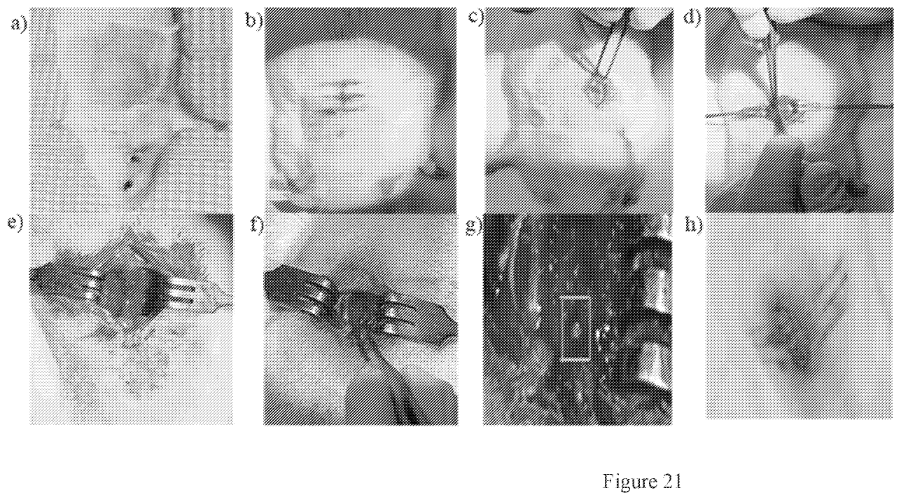

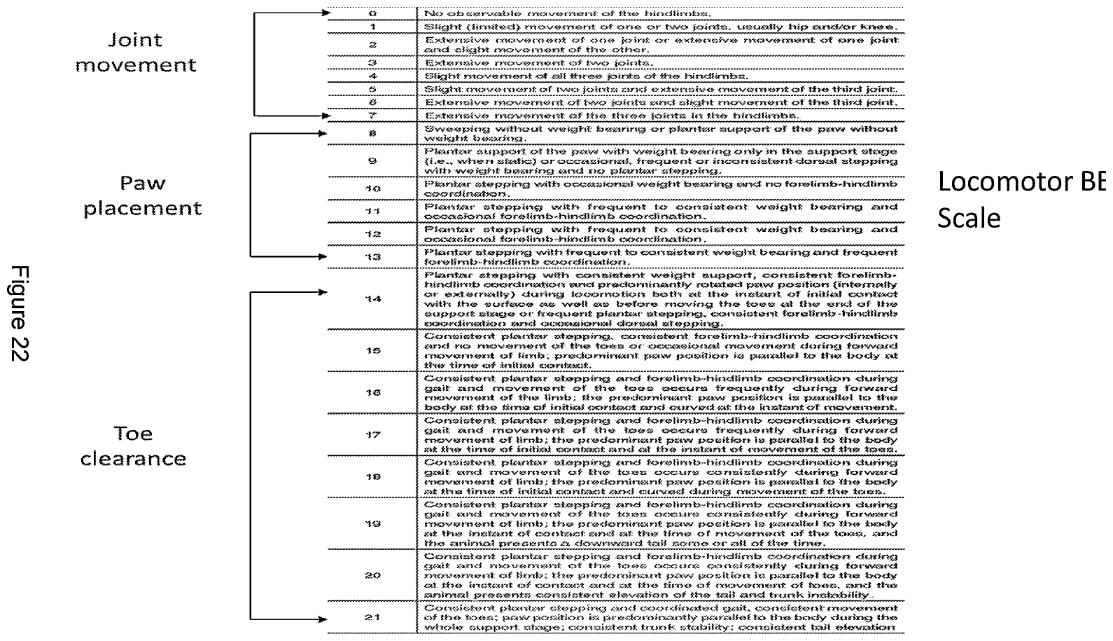

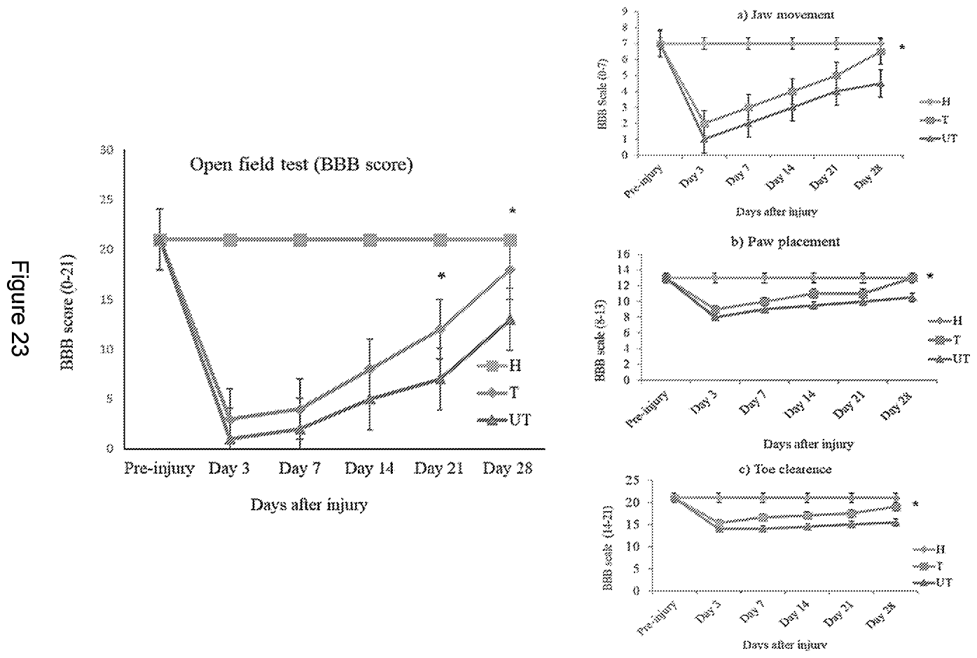

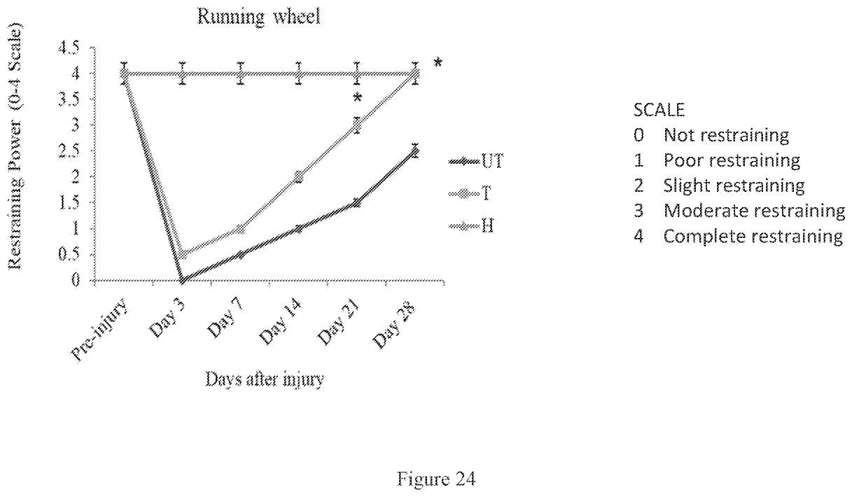

Results: Sprague Dawley rats were divided into three groups (i) SCI rats with MLC901 treatment, (ii) SCI rats without treatment and (iii) healthy rats. The number of rats in each group was five. After injury using the method above, the animals were treated with 10 mg/kg/day of MLC901 in drinking water. If the animal didn't take drug with water, the animals were force fed by oral gavage 16 G. The duration of treatment consisted of 28 days and during these 4 weeks the animal was assessed for their locomotor movement by open field, running wheel, inverted grid and electrophysiology analysis, as shown in FIGS. 23-27. The open field analysis was carried out by two “blinded” people and scored using the BBB scale shown in FIG. 22, reflecting sub-scores for joint movement, paw placement and toe clearance. FIG. 23 shows a line graph of BBB scores and sub-scores of controls (healthy rats), untreated rats and treated rats after mechanical spinal cord injury at day 3, 7, 14, 21 and 28 post-injury. As demonstrated, treated rats showed better BBB scores (p<0.05) for jaw movement, paw placement and toe clearance than untreated rats. FIG. 24 shows a line graph of running wheel restraining power of control (healthy rats), untreated rats and treated rats after mechanical spinal cord injury on day 3, 7, 14, 21 and 28 post-injury on a scale of 0-4, where 0 showed no restraining power and 4 indicated complete restraining power. As demonstrated, treated rats showed better restraining power (p<0.05) than untreated rats, n=5. FIG. 25 shows a line graph of grid walking for control (healthy rats), untreated rats and treated rats after mechanical spinal cord injury on day 3, 7, 14, 21 and 28 post-injury, where a) represents the time for walking a 0.5 m grid and b) is the distance (line crossed) of grid travelled within 60 seconds. As demonstrated, the treatment group showing better grid walking and more line crossed (p<0.05) as compared to untreated rats.

Table 1 below shows the grid walk test results, including the total number of footsteps taken in 1 minute, the number of fore- and hind-limb faults and percentage of total faults for treated and untreated rats after mechanical spinal cord injury at pre-injury, and day 3, 7, 14, 21 and 28 post-injury. As demonstrated, the treatment group at each day tested post-injury showed a statistically significant improved result compared to the untreated group (p<0.05*).

TABLE 1

| | Total no of foot steps | | No of fore limb and | | Total foot faults | |
|---|---|---|---|---|---|---|
| | (1 min) | | hind limbs faults | | (%) | |
| Days | Untreated | Treated | Untreated | Treated | Untreated | Treated |
| PRE-INJURY | 32.66 ± 2.33 | 33.5 ± 1.87 | 3.5 ± 0.54 | 3.0 ± 0.63 | >5 | >5 |
| 3 DAYS | 11.16 ± 1.16 | 14.5 ± 1.37 | 8.83 ± 0.75 | 7.16 ± 0.75 | >10 | >8 |
| 7 DAYS | 17.5 ± 1.37 | 21.3 ± 1.36 | 7.16 ± 0.75 | 5.5 ± 0.54 | >9 | >6 |
| 14 DAYS | 22 ± 0.89 | 25.7 ± 1.03 | 5.5 ± 0.83 | 4 ± 0.89 | >7 | >5 |
| 21 DAYS | 25.2 ± 1.16 | 28.5 ± 1.04 | 4.66 ± 0.51 | 3.16 ± 0.41 | >6 | >5 |
| 28 DAYS | 27.8 ± 0.75 | 31 ± 1.09 | 3.83 ± 0.40 | 2.66 ± 0.51 | >5 | >5 |

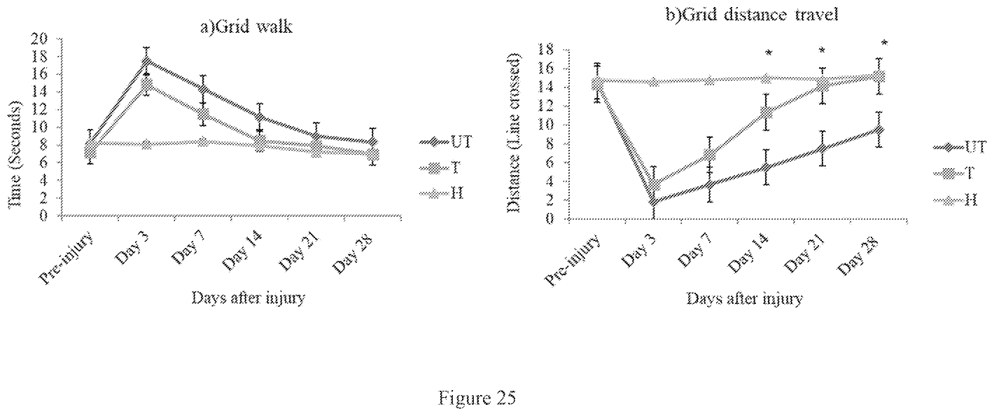

FIG. 26 shows a line graph of an inverted grid test for control, untreated rats and treated rats after mechanical spinal cord injury at pre-injury and days 3, 7, 14, 21 and 28 post-injury, reflecting the time to hold the grid in inverted position, where more time shows more ability and strength of fore- and hind-limbs. As demonstrated, the treatment group was able to hold an inverted position for longer than untreated rats (p<0.05).

Table 2 below shows the results of a sensory function assessment of cold and hot reflexes, where sensory coordination is tested in treated and untreated rats using a hot and cold sensation/scale of from 0-3, where 0 indicates no response, 1 indicates a localized response, 2 indicates transient vocalization and 3 indicates sustained vocalization

TABLE 2

| | |
|---|---|
| Hot sensation | Cold sensation |

| Days | Untreated | Treated | Untreated | Treated |
|---|---|---|---|---|
| Pre-injury | 3 | 3 | 3 | 3 |
| 3 | 0 | 0 | 0 | 1 |
| 7 | 0 | 1 | 1 | 2 |
| 14 | 1 | 2 | 2 | 3 |
| 21 | 2 | 3 | 3 | 3 |
| 28 | 3 | 3 | 3 | 3 |

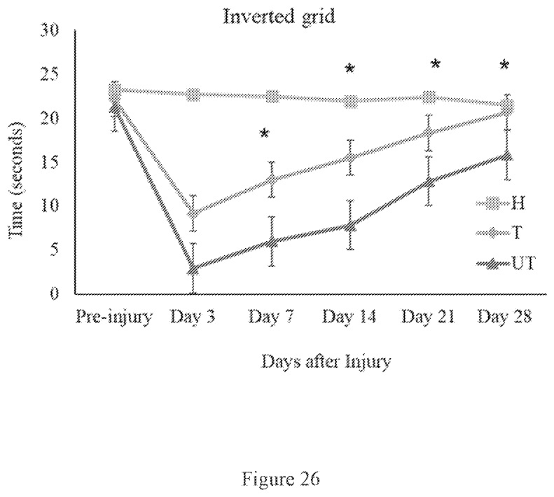

FIG. 27 is a bar graph reflecting the electrophysiology SEP showing the latency, duration, and amplitude in both treated and untreated groups. The latency and duration is increased and while amplitude is decreased in both treated and untreated groups. No significant difference is observed in both groups.

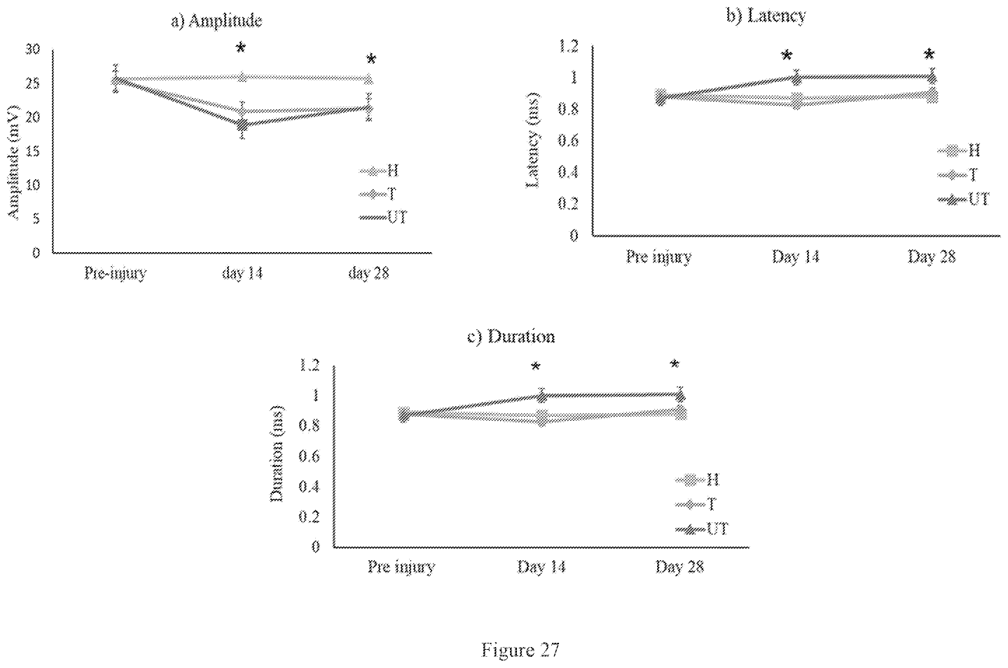

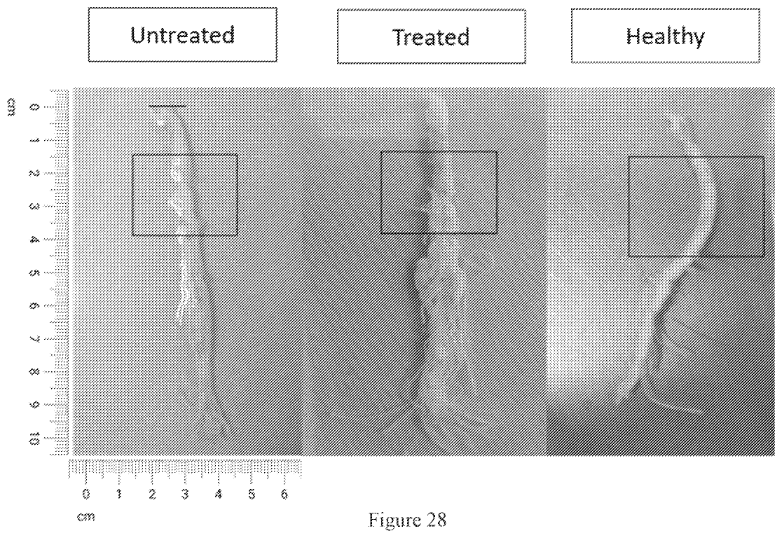

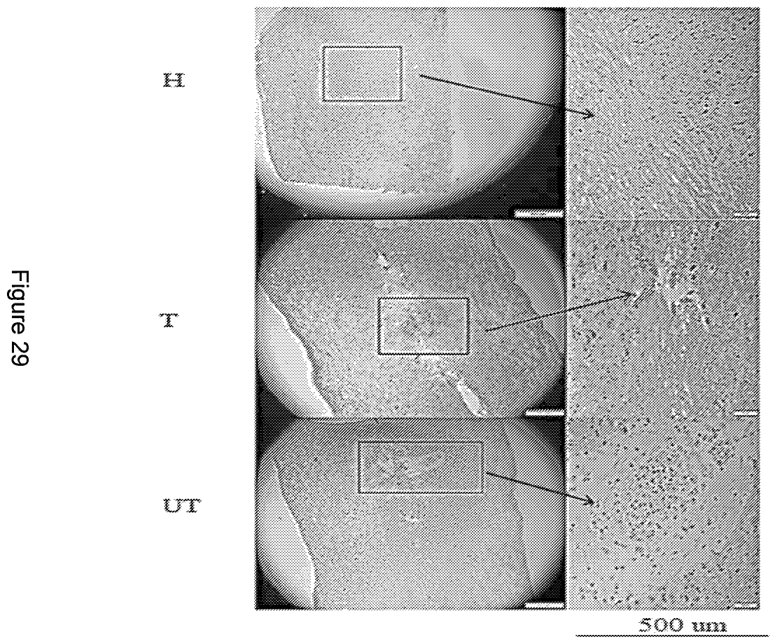

FIG. 28 shows spinal cord morphology after extraction. The spinal cords of untreated, treated and healthy rats are shown in FIG. 28. FIGS. 29 and 30 show histopathological analysis of spinal cord lesions stained with H and E.

Discussion: The mechanical compression injury method successfully induced hind limb paralysis in rats with moderate and incomplete SCI. The calibrated forceps compression method is a convenient and reproducible laboratory method, where the intensity of injury is controlled by the compression duration. The method was used to produce a gait analysis between the MLC901 treatment group, an untreated group and healthy rats in a variety of tests. Following testing, the MLC901 treatment groups regained movement within 2 weeks, while the untreated group took 4 weeks to regain movement. The running wheel, grid walk and inverted grid test results also indicate that the MLC901 treated rats scored significantly better than untreated rats, showing MLC901 helps to recover lost connections between the brain and spinal cord. The SEP result indicates less latency and higher amplitude indicating disruption in potential transmission.

### Objective 3: Establish an In Vivo Chemical Spinal Cord Injury Model in Adult

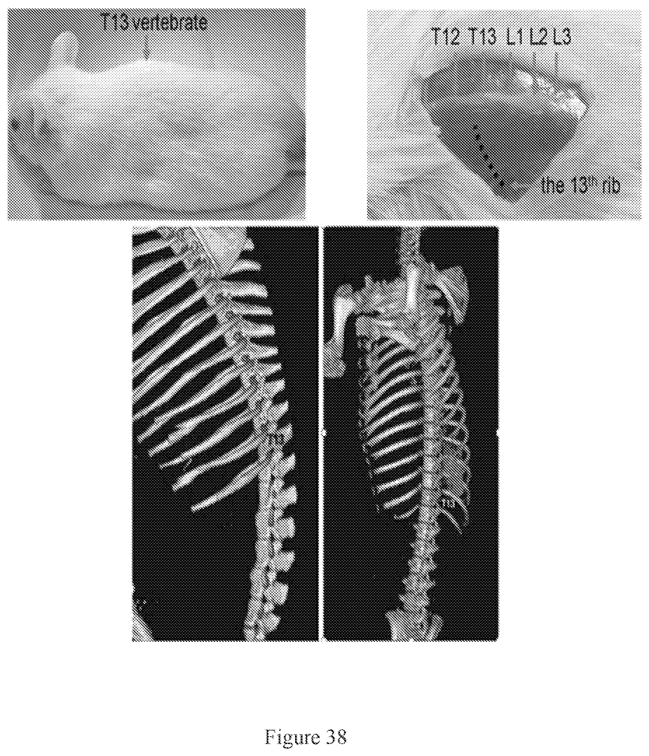

Method: Fifteen (15) adult Sprague Dawley (SD) rats (weighing 300 g to 400 g) were housed in clean cages under a bio-bubble air control system and maintained in a 12-hour light-dark cycle with access to food and water ad libitum. The rats were acclimatized to the environment 7 days prior to the experiment. The rats were anesthetized by administering ketamine-xylazine (9:1) solution via intramuscular injection at 0.1 mL/100 g animal body weight. Once the rats were anesthetized, ointment was applied to the eyes to prevent dehydration, and the rats were placed on a heating pad. The dorsal surface was shaved and the vertebrae were marked from the T11-T13 positions approximately 1 cm around the intended incision location. The T13 vertebra was determined by palpating the 13th rib externally, then the vertebra was visualised with forceps. The forceps were hooked gently underneath the rib to determine to which vertebra it was attached, i.e. the T13 vertebra will move in response to the rib movement. By counting upward the T11 vertebra was identified. The incision site was disinfected three times with 70% isopropyl alcohol, then with iodine solution. FIG. 38 depicts the anatomical location of the T13 thoracic spinal cord segment in a top-down view of the back of the SD rat. The skin was opened to expose the vertebral column at T12-L3. T13 is adjacent to the 13th rib.

Before making the incision, the rats were checked for reflexes using the toe or tail pinch method to ensure they were properly anesthetised. Then, an incision was made along the dorsal spine, and the rat's reflexes were checked again. A line was drawn along the T11-T13 vertebrae and the skin was cut through approximately 1.5 cm. A retractor was inserted to hold the skin and the tissue was cleared on either side of the spinal cord to locate the T13 vertebra. With proper lighting, the space between the T12 and T13 vertebrae was determined. The needle was slowly inserted between the vertebrae, and reflexes in the lower limb and tail were checked for proper positioning.

The KA (1 mL, 0.1 mM) was diluted with 1 mL normal saline to produce a final concentration of 0.05 mM KA, of which 40 μL was injected at the rate of 0.01 mL (10 μL)/min until the syringe was empty. The lower limbs and tail stiffness that followed rapidly after the injection demonstrated successful injury induction. The rats were placed in an upright position for 3-5 min after the KA injection to prevent drug perfusion to the brain, and then gently placed in a supine position on a heating pad. Then, gentle pressure was applied with a surgical sponge to halt the bleeding, taking care not to apply pressure to the spinal cord.

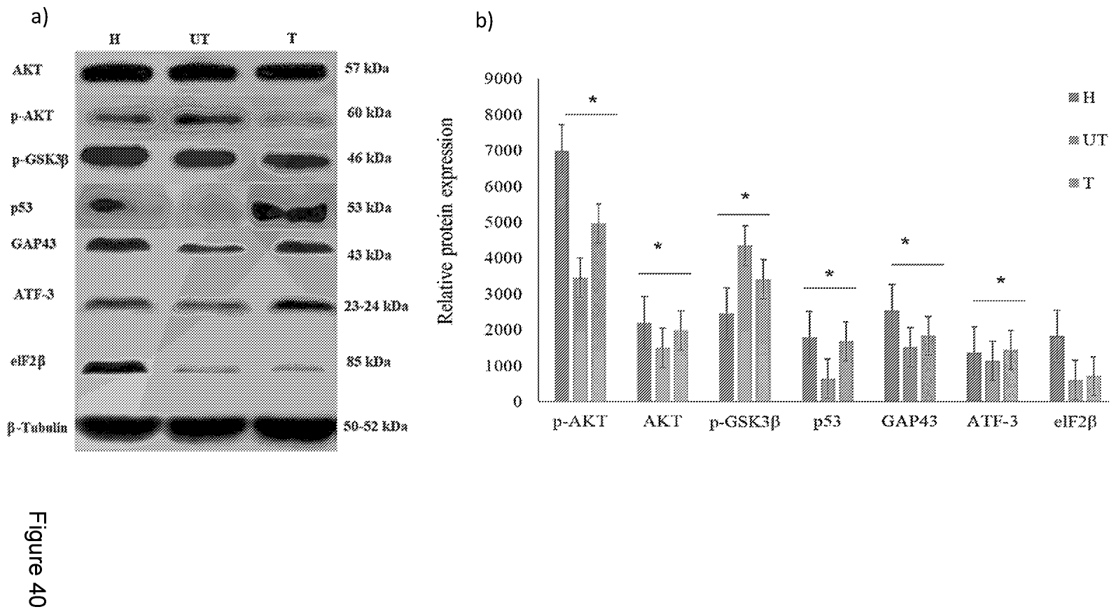

The muscle layer was carefully sutured over the spinal cord, taking care not to disrupt or apply pressure on the spinal cord. The skin was closed over the wound using sutures and the rats were kept on a ventilator and heating pad until consciousness was regained (60-90 minutes). Subsequently, 3-4 mL Ringer lactate solution/300-400 g body weight (pre-warmed to 37° C.) was injected to avoid dehydration due to the surgery. Tramadol stock solution (0.4 mg/100 g, 50 mg/mL) was administered 5 minutes after wound closure. Povidone and topical antibacterial ointment was applied to the wound. The rats were closely monitored until consciousness was regained, then were transferred to a regular clean cage with comfortable and clean bedding. FIG. 40 provides a diagrammatic representation of KA-induced spinal injury, where: A) shows the marking of T11-T13 vertebrae; B) shows the subcutaneous incision; C) shows the exposure of muscle and spine; D) shows the removal of the muscle layer; E) shows the insertion of 26-G syringe in the vertebral space between T12 and T13 vertebrae; F) shows the syringe placement at 45° angle; G) shows the slow perfusion of KA (0.1 μL/min); and H) shows the wound closure.

After the spinal injury, 0.4 mg/100 g subcutaneous tramadol twice daily for 3-5 days was administered to alleviate pain symptoms. Soft food and autoclaved clean water were provided nearby and easily accessible to the rats. The daily food and water intake was monitored carefully, and if a rat did not feed and drink properly, Ringer lactate solution (1 mL/100 g body weight subcutaneously) was administered 3-5 days post-injury. Urinary retention typically occurs because of lower limb paralysis. To avoid this, the bladder was manually massaged twice daily to facilitate urination: the abdomen was gently palpated to locate the bladder, then gentle downward pressure was applied until the bladder was empty. In case of bloody urination, the antibiotic Baytril (100 mg/mL) at 50 mg/100 g body weight was injected subcutaneously.

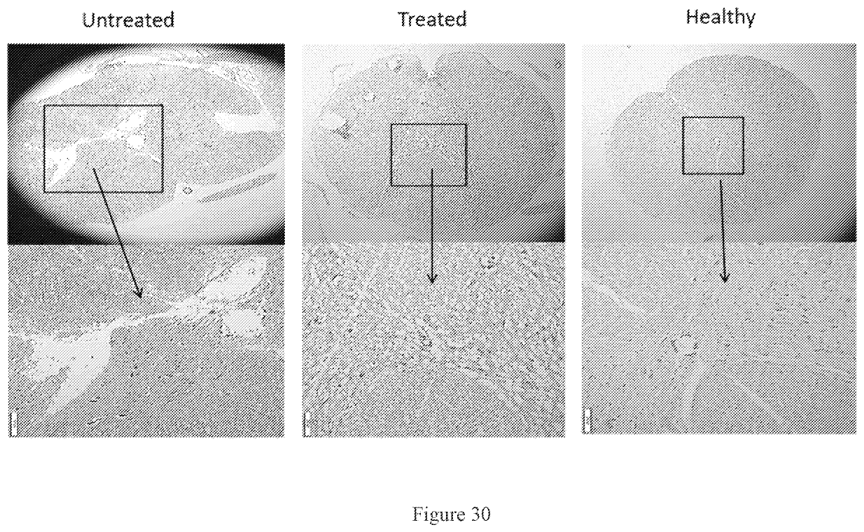

Injury was induced in two of the three groups of rats. After injury, the rats were divided into treated (T), untreated (UT) and healthy (C) groups. The experimental timeframe for the procedures of various assessments is described in FIG. 31. All rats go through the pre-assessment during the week before surgery at day 1, day 3 and day 7. After surgery, the electrophysiological evaluations were conducted at day 0, day 14 and day 28 respectively, while locomotor tests were conducted at day 3, day 7, day 14, day 21 and day 28 respectively. Sensory function tests were carried out on day 7 and day 21. The animals were sacrificed to collect spinal cord specimens at day 28, which were used for histological and pathological studies.

### Objective 4: Investigate the Efficacy of Compositions of the Present Disclosure on Neuronal Regeneration in Rats Whose Spinal Cords are Injured by Chemical Means

Method: The experimental timeframe for the various assessment is described in FIG. 31. All rats undergo a pre-assessment (1 week before surgery) at day 1, day 3 and day 7. After surgery, the electrophysiological evaluations were conducted at day 0, day 14 and day 28, while locomotor tests were conducted on day 3, day 7, day 14, day 21 and day 28. Sensory function tests were carried out at day 7 and day 21. The animals were sacrificed at day 28 and spinal cord specimens were taken, which were used for histological and pathological studies.

### Results:

### Locomotor Activity Assessment

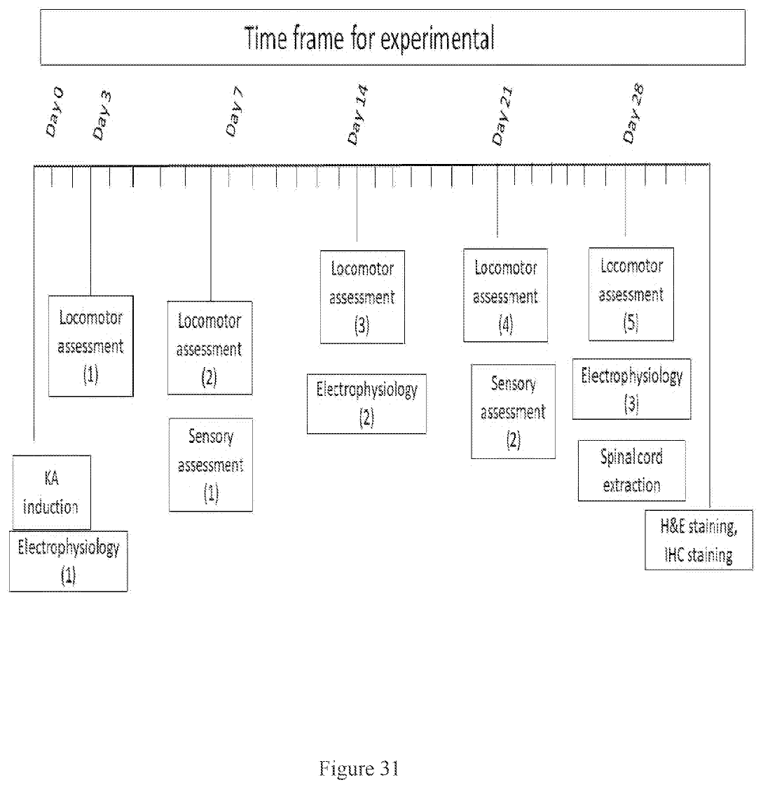

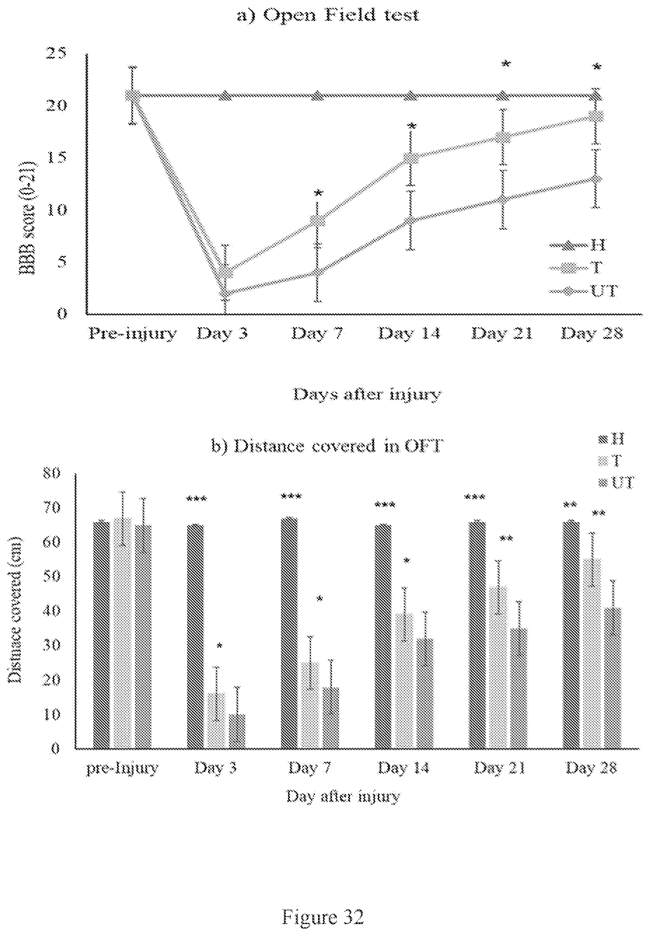

Following the KA injury, the rats exhibited full paraplegia, with no activity in the hind limb or tail, as well as urination dysfunctions without defecation dysfunction. Retraction to the puncture began to heal one week post-injury in both injury groups (T & UT). Slight hind limb movement occurred one week post-injury in the T group and became more pronounced after 3 weeks. However, in the UT group, slight coordinated movement was observed only after day 14. Complete coordinated movement was not seen in either group even after day 28. In the T group, the hind limbs exhibited slightly more coordinated activities after day 28, but in the UT group less improvement was observed [FIG. 32a)]. The urinary function was partially restored after 3 days, although residual urine remained in the bladder and completely resolved after 7-10 days in both injury groups. The BBB scale comprises eight categories that allow for separate evaluation of the forelimbs and hindlimbs: articular movements of the affected limbs, weight support, digit position, paw placement, orientation and movement during stepping, limbs coordination and tail positions. A total maximum score of 21 points indicates normal locomotion or full functional recovery and 0 represents no movement. BBB scores in the C, T and UT groups were scored by two blinded observers. The results showed the UT injured group had significantly lower BBB scores than the T group at day 7, 14, 21 and 28 post-injury [FIG. 32a)].

The distance covered was observed for both pre-SCI and post-SCI rats to examine spontaneous locomotor activity [FIG. 32b)]. The recordings and scoring were taken during the 5 minutes of walking in the Open Field test. The UT injured rats showed significantly reduced distance covered compared to the T group. The calculated distances covered at days 3, 7 and 14 for respectively UT and T rats were 10.5±0.707 cm and 17.5±0.76 cm (p<0.05*), 17.5±0.707 cm and 25±2.707 cm (p<0.05*) and 32.5±2.12 cm and 39.2±3.53 cm (p<0.05*). The distance covered at days 21 and 28 (p<0.01**) between UT and T groups were respectively 35±2.82 cm and 47±1.12 cm and 41±2.207 and 55±2.432. Decreased covered distance in UT rats was matched by hind limb paraplegia and loss of coordination [FIGS. 32a) and 32b)].

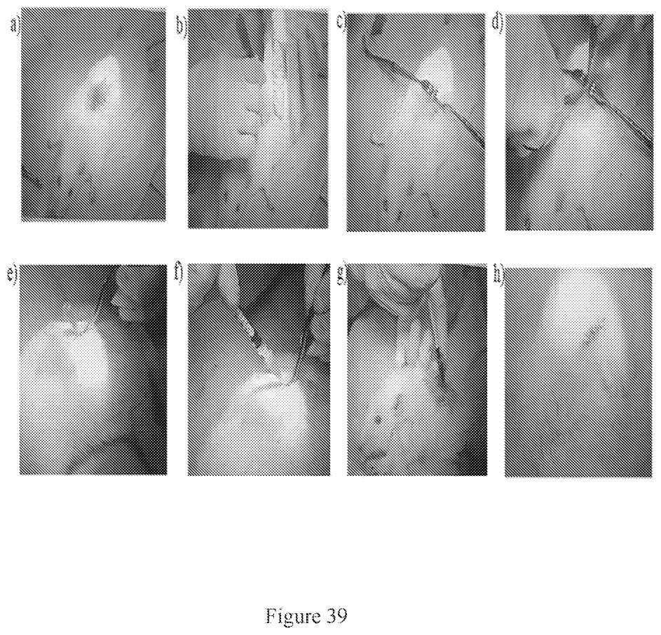

Locomotor gait analysis was also estimated in the Open Field. The movement of the KA-injured rats was compared with that of control (healthy) rats and scored by two blinded observers using the BBB scale pre-injury and at day 7, 14, 21, and 28 post-injury. The scores for jaw movement, jaw placement and toe clearance are shown in FIG. 39.

The running wheel assessment of the pre-injury C, UT and T groups was estimated and compared. UT and T rats showed complete loss of restraining power after injury and showed (p<0.001***) significantly reduced restraining compared to the C group. However, at days 3 and 14 (p<0.05), and at days 21 and 28 (p<0.01), the T group showed greater restraining power than the UT group [FIG. 33a)]. Similarly, the Grid Walking and Inverted Grid assessments demonstrated that both injured groups (UT and T) significantly lost the strength to hold the grid in the inverted position, due to hind limb paralysis, and were unable to walk on the grid without numerous observed foot faults [FIG. 33b), 33c) and 33d)]. However, with progressing days of treatment, the T group showed improvement (p<0.05 and p<0.01), demonstrated by more holding time and grid distance travel than the UT group. This was statistically significant at days 7, 14, 21 and 28 post-injury (FIG. 33).

### Sensory Function Test

Sensory functions were carried out at days 7 and 21 after KA injury. The hot and cold sensation test indicated that, after the injury, both UT & T group rats did not show any response at day 3, while at day 7 UT rats were scored 0 by both observers whereas T group rats showed slow withdrawal and were scored 1 (see Table 3 below). The re: showed delayed response and indicated impaired sensory nerve conduction. However, pre-injury and C group rats received a score of 3 throughout the experiment with quick withdrawal. The latency of the withdrawal response (the time between stimulation to the withdrawal of the hind paw) of the KA injured rats was shorter than that of the C group rats. The latency of the withdrawal response observed for T rats was better from day 7 than UT rats (see Table 3).

TABLE 3

| | |
|---|---|
| Hot sensations | Cold sensations |

| DAYS | UT | T | UT | T |
|---|---|---|---|---|
| Pre-injury | 3 | 3 | 3 | 3 |
| 3 | 0 | 0 | 0 | 0 |
| 7 | 0 | 1 | 0 | 1 |
| 14 | 1 | 2 | 1 | 2 |
| 21 | 2 | 3 | 2 | 3 |
| 28 | 3 | 3 | 3 | 3 |

### Electro-Physiological Assessment

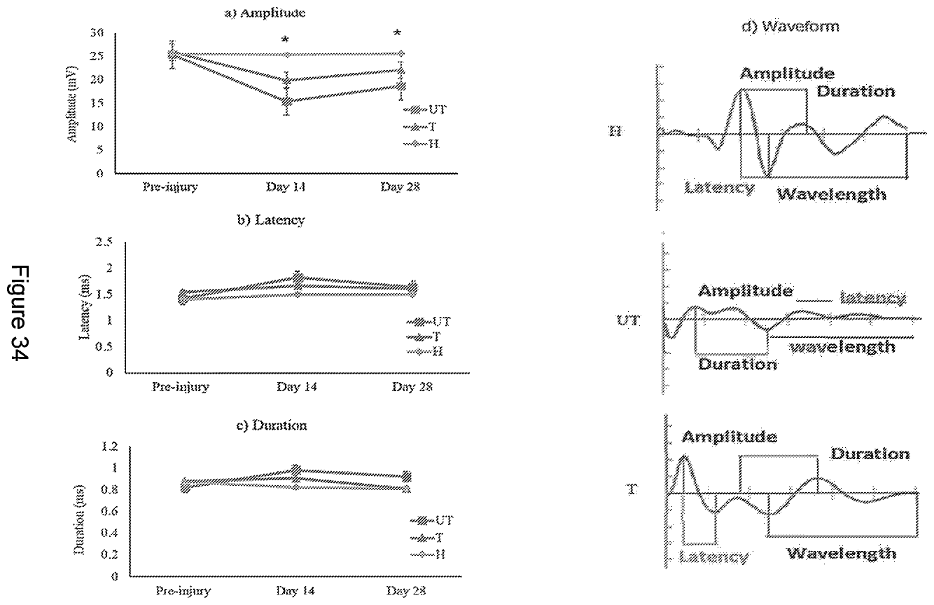

SEPs testing results showed that the waveforms disappeared just after KA-induced SCI in both injury groups. The electrophysiology results correlated with the results of locomotor assessment. The healthy group showed no significant change in the SEPs waveform over time. In contrast, in KA injured groups (UT and T) the SEPs waveform disappeared immediately after SCI and no waveform were observed even 30 minutes after injury. FIG. 34 a), b), c) and d) showed the waveform, mean onset amplitude, duration and latency respectively of both UT and T groups at days 14 and 28. A decrease in the amplitude was observed in UT and T injured groups as compared to pre-injury and the C group. The mean onset amplitudes in the combined KA injured groups were 19.56±1.52 mV on day 14 and 22.3±0.81 mV on day 28. The mean onset amplitudes in the C group on days 0, 14 and 28 were 25.63±0.81 mV, 25.67±0.50 mV and, 26.02±1.14 mV [FIG. 34b)]. The UT injured group showed statistically (p<0.05) reduced amplitude at day 14 compared to the T group and statistically (p<0.01) reduced amplitude at day 14 compared to C group [FIG. 34b)]. The duration of nerve conduction demonstrates the nerve conduction velocity. It was observed that the duration was increased significantly (p<0.05) at Day 14 in the UT group (1.32 0.11 ms) compared to the T group (0.42±0.17 ms) [FIG. 34a) and c)]. Similarly, a statistically significant (p<0.05) increase in latency was observed after day 14 post-injury in the UT group compared to the T group. There was no waveform observed for somatosensory evoked potential at day 0 just after injury. However, after day 14 UT rats hindlimbs exhibited severe motor dysfunction, as reflected by the amplitude which decreased sharply, and duration and latency, which were significantly increased compared to the T group [FIG. 34 a), b), c) and d)]. Histological analysis

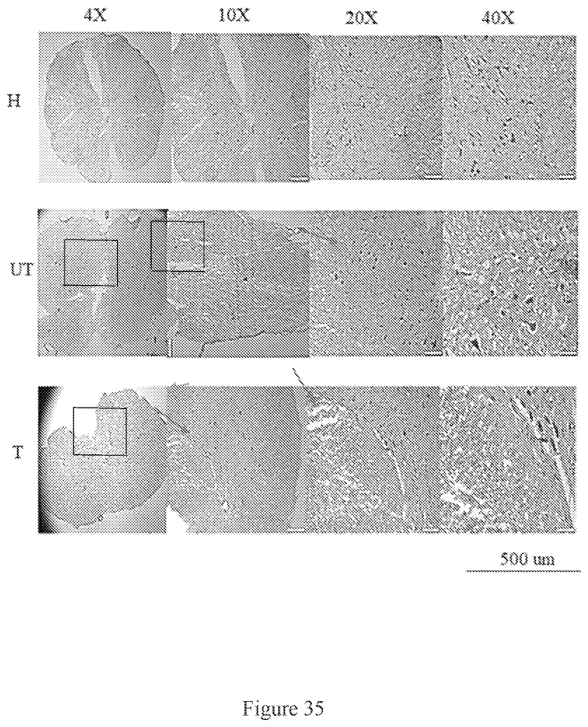

Tissue damage was observed on the spinal cord transverse sections of KA injured (UT and T) rats. A histopathology study using H and E staining on the spinal cord tissue showed the appearance of lesions at the KA injection site. The most severe damage was at the epicentre, with loss of the ventral horns of grey matter and lateral funiculus of white matter (see FIG. 35). H and E staining results indicated severe structural damage and decreased neuronal cell numbers in both UT and T group as compared to the C group. The presence of hemorrhagic foci was observed in the middle of the grey matter. The lesion area in the middle (grey matter) showed the progressive necrosis and cavitation. A number of erythrocytes and neutrophils had emerged at the primary lesion site in both UT and T rats. The UT rats showed bigger hemorrhagic foci than T rats, which indicated more damage and nerve degeneration. Significant albumin extravasation was observed in the spinal cord sections of UT and T KA injured rats. However, no albumin extravasation was observed in C group. The presence of albumin extravasation in KA injured rats was primarily caused by neurodegenerative and glutamate excitotoxicity. This colocalization effect of albumin extravasation in neuronal cells indicated more neurotoxicity and neuronal cell death in UT rats than T rats (see FIG. 35).

### Immunohistochemical Analysis

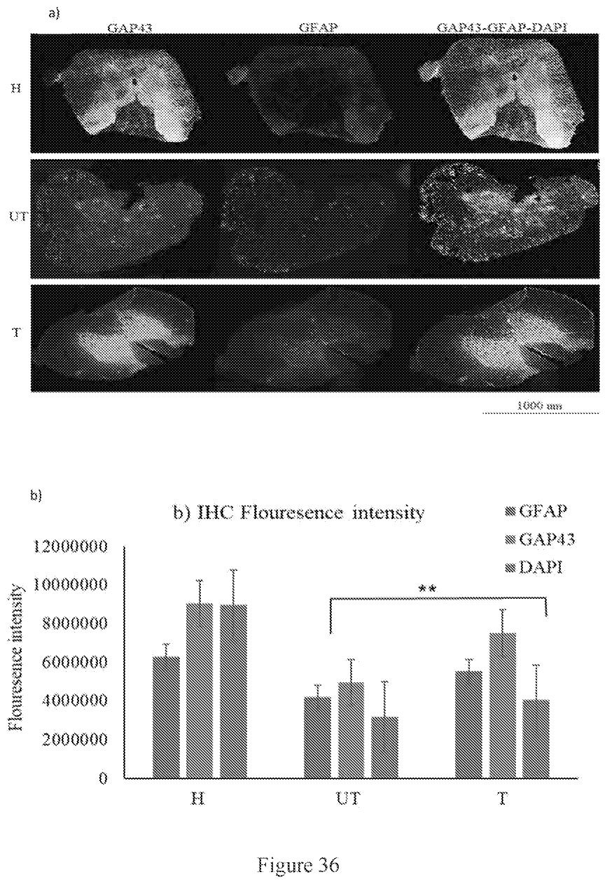

Macroscopic evaluation of dissected spinal cords revealed striking hemorrhage and tissue damage at the lesion site in KA injured group. Immunohistochemistry showed systematic expression of GAP-43 and GFAP in both UT and T groups. Highly downregulated GAP43 expression in the UT and T groups revealed close proximity to the lesion formation and neuronal degeneration (see FIG. 36). GAP-43-positive neuronal fibers were found in the grey matter in the C group. However, lower expression of green fluorescence in grey matter of the UT group compared to the T group reflected neuronal degeneration. Double staining with GFAP also showed astrocyte presentation in C, UT and T groups. Less GFAP expression indicated astrocyte loss as well. The confocal images reflect significant differences in the expression of GAP-43 and GFAP between the C, UT and T groups (see FIG. 36).

Discussion Following KA intra-spinal administration, male SD rats displayed major signs of motor dysfunction and sensory impairment. Behavioral changes, such as anxiety and a tendency to remain seated at the corner of the cage, were observed in the KA injured rats, basically due to glutamate excitotoxicity. Rats that remained stationary for longer than 15-20 seconds were forced to move by lightly tapping or scratching on the side of the Open Field. If the animal still did not respond to these actions, it was picked up and placed in the center of the Open Field apparatus, which caused it to move toward the other side. Sometimes, the observation period was extended beyond 5 minutes for a more accurate assessment of toe clearance, paw position or forelimb-hindlimb coordination.

The BBB scale is an excellent tool to access locomotor activities. The lowest score “0 with no observable hindlimb (HL) movement”, usually was given in severe SCI conditions. UT rats in this study showed the lowest BBB scores of 3 to 4 points indicating moderate SCI. The T rat group reached up to 19 points after day 28 following injury. Two blinded observers rated the behavior from individual joint movements of the hindlimb, to plantar stepping, to coordinated walking, and finally the subtler behaviors of locomotion, such as paw position, trunk stability and tail position. The highest score in the BBB scale is 21 indicating “consistent plantar stepping and coordinated gait” revealing full recovery and balanced gait. UT & T group rats achieved a maximum of 13 and 19 points (day 28 following injury) reflecting motor dysfunction. Only C group and pre-injury rats scored 21 points on the BBB scale. It was also observed that little improvement in the score for UT rats was seen between days 21 and 28 post-injury ensuring. The T group rats showed a short lag phase in the first three days following injury, following by a more rapid phase of recovery between days 4 and 13, followed by a functional plateau between days 15 and 28. The BBB score is important for the interpretation of changes in locomotor activity following spinal cord injury, and is used herein to detect variability in outcomes between UT and T groups.

Rats placed in a running wheel tend to restrain and stop the wheel by the forceful gripping of fore and hind paws, which is why the running wheel test is considered valuable when assessing the strength and coordination of rats after SCI. The BBB scores and distance covered (in cm) by all three groups indicated statistically significant (p<0.05 *and p<0.01**) lower scores in KA injured rats, reflecting successful induction of moderate paraplegia. The result also indicated T rats showed better BBB scores and distance covered than UT group, emphasizing the value of this model to study the regenerative potential of therapeutics under laboratory conditions.

Similarly, the grid walk test is another important laboratory test to evaluate and compare the gait in different rat groups, and to study the regenerative potential of therapeutics under laboratory conditions. In the grid walking test, rats are required to place their limbs accurately in certain places. When the paw of a rat falls, this is considered a foot fault. Errors and faults are counted during walking on the grid, with more faults reflected greater motor coordination defects.

The results obtained by the running wheel and grid walking tests showed the T group rats exhibited significantly better motor coordination than the UT group rats. The finding indicated a substantial improvement of motor control in T group rats over UT group rats [see FIG. 33a), b), c) and d)]. In the inverted grid test, the T group rats showed significantly better power than the UT group rats.

The results demonstrated that the coordination and locomotor disorders associated with KA injury in rats remain persistent until day 28 after KA injury. These motor dysfunctions remain unchanged until day 3 after injury for both UT and T rat groups, when T group rats then started to show more improvement that UT rats up to 28 days, indicating T group rats possess neuro-regenerative potential. The result obtained from all locomotor tests indicate that MLC901 improves the locomotor recovery of injured rats, which may also be associated with increased GAP43 expression (neuronal marker) and less demyelination (H and E). Greater GAP43 expression in T rats than UT rats indicate that MLC901 attenuates the improvement in locomotor activity by restoration of lost connection and neuronal regeneration.

Electrophysiological assessment is reflected as a functional means to evaluate the integrity of various aspects of the nervous system, including the spinal cord. Clinical applications of electrophysiology, particularly evoked potentials such as somatosensory and motor evoked potentials, have been included in the diagnosis of peripheral or central nervous system damage, particularly somatosensory evoked potentials (SEPs) monitoring the integrity of the dorsal columns in the spinal cord [Cruccu et al, Clinical Neurophysiology, 119(8), pages 1705-1719. (2008)]. SEPs provide a reproducible, non-invasive, and objective assessment of axonal conduction of descending sensory and motor pathways in both normal and injured spinal cord [Malhotra & Shaffrey, Spine, 35(25), pages 2167-2179 (2010)]. The pulse conduction through neurons can be measured by stimulating electrical signal from the nerve itself or from a muscle innervated by that nerve. Latency is measured in terms of time from stimulation by stimulating electrodes to the recorded signal deflection by recording electrodes. The time it takes for the electrical impulse to travel from the stimulation site to the recording site is called as the latency. The time required in conduction is known as duration and is measured in milliseconds (ms). The size of the response of the impulse conduction in motor and sensory neurons is known as the amplitude and is measured in millivolts (mv). The signal is recorded as the nerve-conducted that passes under the recording electrode, and latency divided by distance can estimate conduction velocity (meters per second). The measurements can be described in terms of the latency to response onset, the amplitude of the response, and the conduction velocity (duration). In the present study, the SEP waveforms of various groups were observed in term of latency, amplitude and duration by placing electrodes in the sciatic nerve near the knee area of rats. Increases in latency and duration in both injury group (T and UT) reflected both peripheral neuronal demyelinating and neuronal loss. Neuronal loss affects the amplitude of nerve conduction velocity. The demyelination of neurons causes slowing of latency. In this study, both UT and T injury groups showed no waveform just after injury (within 30 min), but a significant (p<0.05) increase in latency in the T group after day 14 following injury compared to the UT group. This demonstrated improvement in nerve conduction following treatment. A decrease in amplitude is directly related to low motor neurons (nerve degeneration) involved in conduction of impulse and presence of demyelinating lesions. Low amplitude and an increase in latency and duration results in slow nerve conduction velocity (NCV), which is indicative of a demyelinating lesion, whereas a low amplitude value is reflective of axonal degeneration. In this study, we found that the no NCV was observed just after injury (30 min). However, T group rats showed better NCV than UT group rats at day 14.

Morphological and pathological interventions are also important in the evaluation of nerve degenerative disorders. The routine H and E staining can show the respective architect of spinal cords after injury and can also detect nerve demyelination in spinal cord sections following SCI. The H and E stained sections after KA injection showed the presence of hemorrhage foci, especially in the grey matter. Progressive necrosis and cavitation were also observed in UT and T KA injured rats (see FIG. 35). However, the hemorrhage and cavitation in the UT group were more pronounced than in the T group, where cavity formation was reduced following treatment with MLC901 (FIG. 35). Inflammatory cells, such as erythrocytes and neutrophils, were more present in the UT and T groups than were observed in the C group, showing nerve degeneration caused by KA excitotoxicity (FIG. 35).

The expression of GAP43 and GFAP were determined to ensure neuronal mass in all groups, i.e C, T and UT. Growth-associated protein 43 (GAP43) is an activity-dependent plasticity protein enriched in axons and neurons to promote actin polymerization and axon regeneration. The Glial Fibrillary Acidic Protein (GFAP) is the main structural protein of the filaments within the cytoskeleton of astrocytes and acts as a marker of mature astrocytes. The results obtained indicated GAP43 is highly expressed in the C group, but less so in the injured groups. It was demonstrated in this study that GAP43-positive fibers were significantly increased in the T group (see FIG. 36), indicating that treatment with MLC901 may have supported neuronal and axon regeneration. It was also observed that GFAP expression was at a higher level in the T group than in the UT group, supporting the fact that treatment with MLC901 can protect astrocyte loss (FIG. 36). However, more expression of GFAP staining in the grey matter indicated the presence of glial and astrocytic scarring in both UT and T groups (FIG. 36).

To summarize, the neurodegenerative outcomes of KA induced injury, and neuro-regeneration in MLC901-treated rats, were observed by locomotor assessments (i.e. Open Field test, running wheel, grid walk and inverted grid), assessing sensory functions and SEPs. The results found following H and E and IHC staining assessed the presence of pathological features. All these results certify the success of the KA excitotoxicity model as an in vivo model to create and study SCI. Moreover, the neuro-regenerative potential of MLC901 was demonstrated by an improvement in all locomotor activity scores, sensory, electrophysiological and histological assessments.

### CONCLUSIONS

The in vivo KA-induced excitotoxicity model offers a practical, convenient and efficient approach to induce SCI in laboratory animals. All the essential finding of this study support the presence of the most prominent physiological and pathological outcomes of SCI. KA intraspinal administration ameliorates the negative impact of injury, causing incomplete paraplegia in rats. The main mechanism of action of KA excitotoxicity is orchestrated by the activation of glutamate receptors that cause more Ca⁺² influx into nerve cell, resulting in ROS and RNS formation and finally stimulates nerve demyelination and neuro-degeneration. Importantly, this study demonstrated that treatment of KA-injured rats with MLC901 improved the functional recovery process of SCI relative to untreated (UT) rats.

### INCORPORATION BY REFERENCE

All references, articles, publications, patents, patent publications, and patent applications cited herein (“References”) are incorporated by reference in their entireties for all purposes. However, mention of any References is not, and should not, be taken as acknowledgment or any form of suggestion that they constitute valid prior art or form part of the common general knowledge in any country in the world. References include the following:
a) WO 2017/048191A1
b) WO 2007/106049A1
c) WO 2010/053456A1
d) WO 2010/110755A1
e) WO 2013/141818A1
f) Cruccu et al., Clinical Neurophysiology, 119(8), pages 1705-1719. (2008)
g) Malhotra & Shaffrey, Spine, 35(25), pages 2167-2179 (2010)

1. A method of treating a subject to induce and/or accelerate recovery from a spinal cord injury comprising administering to the subject an effective amount of a herbal composition comprising at least the four herbal components Radix polygalae (root of thin leaf milkwort), Radix astragali (root of membranous milkvetch), Rhizome Ligusticum chuanxiong and Radix Angelica sinensis (root of Chinese angelica) or extracts thereof.

2. The method as claimed in claim 1, wherein the herbal composition further comprises at least one herbal component selected from the group consisting of Radix et Rhizome salviae Miltiorrhizae (red sage root), Radix paeoniae rubra (red peony root), flower of Carthamus tinctorius (safflower), Semen persicae (Prunus persica seed) and Rhizome Acori tatarinowii (rhizome of grassleaf sweetflag) or an extract thereof.

3. The method as claimed in claim 1, wherein the herbal composition consists essentially of at least the four herbal components Radix polygalae (root of thin leaf milkwort), Radix astragali (root of membranous milkvetch), Rhizome Ligusticum chuanxiong and Radix Angelica sinensis (root of Chinese angelica) or extracts thereof.

4. The method as claimed in claim 2, wherein the herbal composition consists essentially of at least the nine herbal components Radix polygalae (root of thin leaf milkwort), Radix astragali (root of membranous milkvetch), Rhizome Ligusticum chuanxiong, Radix Angelica sinensis (root of Chinese angelica), Radix et Rhizome salviae Miltiorrhizae (red sage root), Radix paeoniae rubra (red peony root), flower of Carthamus tinctorius (safflower), Semen persicae (Prunus persica seed) and Rhizome Acori tatarinowii (rhizome of grassleaf sweetflag) or extracts thereof.

5. The method as claimed in any one of claim 1, wherein
the herbal composition is a pharmaceutical composition.

6. The method as claimed in claim 5, wherein the pharmaceutical composition also comprises a pharmaceutically acceptable carrier or excipient.

7. The method as claimed in claim 6, wherein the pharmaceutical composition is the composition MLC901.

8. The method as claimed in claim 6, wherein the pharmaceutical composition is the composition MLC1501.

9. The method as claimed in claim 1, wherein
recovery from spinal cord injury is induced and/or accelerated by neuronal cell regeneration.

10. The method as claimed in claim 1, wherein said herbal composition promotes the growth of neuronal cells.

11. The method as claimed in claim 10, wherein the neuronal cells are vertebrate neuronal cells.

12. The method as claimed in claim 11, wherein the vertebrate neuronal cells are human neuronal cells.

13. The method as claimed in claim 12, wherein the neuronal cells are derived from cortical neuronal cells.

14. The method as claimed in claim 1, wherein administration of the herbal composition results in spinal cord regeneration.

15. The method as claimed in claim 1, wherein administration of the herbal composition results in regeneration of a connection between brain and spinal cord.

16. The method as claimed in claim 1, wherein administration of the herbal composition results in regeneration of damaged neuronal tissue or cells.

17. The method as claimed in claim 1, wherein administration of the herbal composition promotes limb motion recovery in paralyzed subjects.

18. The method as claimed in claim 1, wherein an effective amount of the herbal composition is from about 1 mg/kg to about 100 mg/kg.

19. The method as claimed in claim 1, wherein the method comprises administering a second agent used for treating a subject with spinal cord injury.

20. The method as claimed in claim 19, wherein the second agent is a pharmaceutical drug effective to control pain and/or muscle spasticity.
